SEQUENCE LISTING (22 sequences)

SEQ ID NO: 1 - protein, 450 aa, Lactobacillus pentosus
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYIGTELVEAYQKQGKEVTLIDGLPRILNKYLDKGFTDRVEKDFVDHGIKMALNQMVKGFSDDGKEVTVKTDKGSYTADMAILCVGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 2 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYSGTELVEAYQKQGKEVTLIDGLPRILNKYLDKGFTDRVEKDFVDHGIKMALNQMVKGFSDDGKEVTVKTDKGSYTADMAILCVGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 3 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYSGTELVEAYQKQGKEVTLIWGLPRILNKYLDKGFTDRVEKDFVDHGIKMALNQMVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 4 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYSGTELVEAYQKQGKEVTLIWGHPRILNKYLDKGFTDRVEKDFVDHGIKMALNCWVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 5 - protein, 450 aa, synthetic construct; identical to SEQ ID NO: 4

SEQ ID NO: 6 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYSGTELVEAYQKQGKEVTLIWELPRILNKYLDKGFTDRVEKDFVDHGIKMALNQMVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 7 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYSGTELVEAYQKQGKEVTLIWELPRILNKYLDKGFTDRVEKDFVDHGIKMALNQMVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRHEFMPTTKPVLMQLVYNPETREILGAQFMSEHDLSQSANVISVMIQNHNTIDDLGFVDMFFQPIYDRPFNYLNLLGQAAIAHAAEKVTE

SEQ ID NO: 8 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYIGTELVEAYQKQGKEVTLIWGLPRILNKYLDKGFTDRVEKDFVDHGIKMALNQMVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 9 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYIGTELVEAYQKQGKEVTLIWELPRILNKYLDKGFTDRVEKDFVDHGIKMALNQMVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRPEFMPTTKPVLMQLVYNPETREILGAQFMSEHDVSQSANVISVMIQNHNTIDDLGFVDMFFQPIYDRPFNYLNLLGQAAIAHAAEKVTE

SEQ ID NO: 10 - protein, 450 aa, synthetic construct
MKVIVIGCTHAGTAAVNQILASNPETDVTIYERNDNVSFLSCGIALYLGGEVADPQGLFYSSPEQLAKLGANVHMQHDVTDVDTENHEITVTDLKTGESKKDYYDKLVVTTGSWPVIPPIDGIDSPNVYLCKNWTHAQSLWEAAKPAKRVIVIGGGYIGTELVEAYQKQGKEVTLIWELPRILNKYLDKGFTDRVEKDFVDHGIKMALNQMVKGFSDDGKEVTVKTDKGSYTADMAILCLGFRPNTSLLKGKVDMNPNGSIKTNDYMQTSDPDIYGAGDSVAVHYNPTKKDAYIPLATNAVRQGTLVGLNIFKPTRKYMGTQSTSGLMLFGKTIVSSGMTLEHAQAEKVPAEAVTFEDNYRHEFMPTTKPVLMQLVYNPETREILGAQFMSEHDLSQSANVISVMIQNHNTIDDLGFVDMFFQPIYDRPFNYLNLLGQAAIAHAAEKVTE

SEQ ID NO: 11 - protein, 261 aa, synthetic construct
MYPDLKGKVVAITGAAEGLGKAMAIRFGKEQAKVVINYQSNKQDPNEVKEEVIKAGGEAVVVQGDVTKEEDVKNIVQTAIKEFGTLDIMINNKGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPRNAEKFADPKQKADVESMIPMGYIGEPEEIAAVAAWLASKEASYVTGITLFADGGMTQYPSFQAGRG

SEQ ID NO: 12 - protein, 249 aa, Francisella tularensis
MKIVKDFSPKEYSQKLVNWLSDSCMNYPAEGFVIGLSGGIDSAVAASLAVKTGLPTTALILPSDNNQHQDMQDALELIEMLNIEHYTISIQPAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 13 - protein, 470 aa, Francisella tularensis
MSFDNLLLMTDSYKHSHRYQYPRDTHYLHFYLESRGTANKDLGNYTKFFGLQYYVKKYLSQPITQQMIDDAEKILLAHGLPFYRSGFEKILNNYNGYLPIRIRAVREGSLIPLHNVLMTIESTDEELFWLPGFVETLLLKVWYPTTVATISFNIKQLIKRYLLETADSLDKLDFMLHDFGYRGVSSEESAGIGGAAHLTNFLGTDTLAALHVCKEFYAEDMAGFSIPASEHSTMTSWGVGTECEREAFENMIAQFGDSSVLYACVSDSWDFKKAIQTWVDLKDRVTAKKANLVIRPDSGDAVDNILYALYELDKGYGSRLNSKGYKVLNNVALIQGDSVSISLAKKVLEAMKIQGYSAENIAFGMGGALLQGNYESSINRDSFKFAIKCSAIMRGNTLIGVKKEPITDLAKKSKQGRLDLIKDAKGNYKTIVLDDSYALGEYHPESQLQYYDNGEIKFEQSLAQIRNYTN

SEQ ID NO: 14 - protein, 473 aa, Zymomonas mobilis
MSSESSQGLVTRLALIAAIGGLLFGYDSAVIAAIGTPVDIHFIAPRHLSATAAASLSGMVVVAVLVGCVTGSLLSGWIGIRFGRRGGLLMSSICFVAAGFGAALTEKLFGTGGSALQIFCFFRFLAGLGIGVVSTLTPTYIAEIAPPDKRGQMVSGQQMAIVTGALTGYIFTWLLAHFGSIDWVNASGWCWSPASEGLIGIAFLLLLLTAPDTPHWLVMKGRHSEASKILARLEPQADPNLTIQKIKAGFDKAMDKSSAGLFAFGITVVFAGVSVAAFQQLVGINAVLYYAPQMFQNLGFGADTALLQTISIGVVNFIFTMIASRVVDRFGRKPLLIWGALGMAAMMAVLGCCFWFKVGGVLPLASVLLYIAVFGMSWGPVCWVVLSEMFPSSIKGAAMPIAVTGQWLANILVNFLFKVADGSPALNQTFNHGFSYLVFAALSILGGLIVARFVPETKGRSLDEIEEMWRSQK

SEQ ID NO: 15 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 16 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 17 - protein, 468 aa, Escherichia coli
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 18 - protein, 165 aa, Escherichia coli
MTDSELMQLSEQVGQALKARGATVTTAESCTGGWVAKVITDIAGSSAWFERGFVTYSNEAKAQMIGVREETLAQHGAVSEPVVVEMAIGALKAARADYAVSISGIAGPDGGSEEKPVGTVWFAFATARGEGITRRECFSGDRDAVRRQATAYALQTLWQQFLQNT

SEQ ID NO: 19 - protein, 410 aa, Escherichia coli
MSSFDYLKTAIKQQGCTLQQVADASGMTKGYLSQLLNAKIKSPSAQKLEALHRFLGLEFPRQKKTIGVVFGKFYPLHTGHIYLIQRACSQVDELHIIMGFDDTRDRALFEDSAMSQQPTVPDRLRWLLQTFKYQKNIRIHAFNEEGMEPYPHGWDVWSNGIKKFMAEKGIQPDLIYTSEEADAPQYMEHLGIETVLVDPKRTFMSISGAQIRENPFRYWEYIPTEVKPFFVRTVAILGGESSGKSTLVNKLANIFNTTSAWEYGRDYVFSHLGGDEIALQYSDYDKIALGHAQYIDFAVKYANKVAFIDTDFVTTQAFCKKYEGREHPFVQALIDEYRFDLVILLENNTPWVADGLRSLGSSVDRKEFQNLLVEMLEENNIEFVRVEEEDYDSRFLRCVELVREMMGEQR

SEQ ID NO: 20 - protein, 363 aa, Pseudomonas putida
MSALFEPYTLKDVTLRNRIAIPPMCQYMAEDGLINDWHQVHYASMARGGAGLLVVEATAVAPEGRITPGCAGIWSDAHAQAFVPVVQAIKAAGSVPGIQIAHAGRKASANRPWEGDDHIGADDARGWETIAPSAIAFGAHLPNVPRAMTLDDIARVKQDFVDAARRARDAGFEWIELHFAHGYLGQSFFSEHSNKRTDAYGGSFDNRSRFLLETLAAVREVWPENLPLTARFGVLEYDGRDEQTLEESIELARRFKAGGLDLLSVSVGFTIPETNIPWGPAFMGPIAERVRREAKLPVTSAWGFGTPQLAEAALQANQLDLVSVGRAHLADPHWAYFAAKELGVEKASWTLPAPYAHWLERYR

SEQ ID NO: 21 - protein, 518 aa, Lactobacillus brevis
MHHHHHHSSGLVPRGSGMKETAAAKFERQHMDSPDLGTDDDDKAMADIGSMKVTVVGCTHAGTFAIKQILAEHPDAEVTVYERNDVISFLSCGIALYLGGKVADPQGLFYSSPEELQKLGANVQMNHNVLAIDPDQKTVTVEDLTNHAQTTESYDKLVMTSGSWPIVPKIPGIDSDRVKLCKNWAHAQALIEDAKEAKRITVIGAGYIGAELAEAYSTTGHDVTLIDAMARVMPKYFDADFTDVIEQDYRDHGVQLALGETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGLNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNAGGSGGSGGSMDYKDDDDK

SEQ ID NO: 22 - protein, 518 aa, synthetic construct
MHHHHHHSSGLVPRGSGMKETAAAKFERQHMDSPDLGTDDDDKAMADIGSMKVTVVGCTHAGTFAIKQILAEHPDAEVTVYERNDVISFLSCGIALYLGGKVADPQGLFYSSPEELQKLGANVQMNHNVLAIDPDQKTVTVEDLTNHAQTTESYDKLVMTSGSWPIVPKIPGIDSDRVKLCKNWAHAQALIEDAKEAKRITVIGAGYIAAELAEAYSTTGHDVTLIARSARVMRKYFDADFTDVIEQDYRDHGVQLALGETVESFTDSATGLTIKTDKNSYETDLAILCIGFRPNTDLLKGKVDMAPNGAIITDDYMRSSNPDIFAAGDSAAVHYNPTHQNAYIPLATNAVRQGILVGKNLVKPTVKYMGTQSSSGLALYDRTIVSTGLTLAAAKQQGLNAEQVIVEDNYRPEFMPSTEPVLMSLVFDPDTHRILGGALMSKYDVSQSANTLSVCIQNENTIDDLAMVDMLFQPNFDRPFNYLNILAQAAQAKVAQSVNAGGSGGSGGSMDYKDDDDK

(19) United States
(12) Patent Application Publication
(10) Pub. No.: US 2026/0357222 A1
(43) Pub. Date: Nov. 12, 2026

(54) GROWTH-BASED SELECTION PLATFORM FOR NONCANONICAL COFACTOR OXIDIZING ENZYMES

(71) Applicant: The Regents of the University of California, Oakland, CA (US)

(72) Inventor: Han Li, Irvine, CA (US)

(21) Appl. No.: 19/124,847

(22) PCT Filed: Nov. 3, 2023
(86) PCT No.: PCT/US2023/078752

§ 371 Date: Apr. 25, 2025

Related U.S. Application Data
(60) Provisional application No. 63/422,767, filed on Nov. 4, 2022.

(51) Int. Cl. C12N 9/04; C07K 14/195; C12N 9/00; C12N 9/12; C12N 9/80; C12N 9/92; C12N 15/70; C12Q 1/02

(52) U.S. Cl. CPC C12N 9/0006; C07K 14/195; C12N 9/1241; C12N 9/80; C12N 9/92; C12N 9/93; C12N 15/70; C12Q 1/02; C12Y 101/01044; C12Y 101/01047; C12Y 101/01049; C12Y 207/07001; C12Y 305/01042; C12Y 503/01009; C12Y 603/01005

(57) ABSTRACT

Provided herein is a growth selection platform for noncanonical cofactor oxidizing enzymes, the noncanonical cofactor oxidizing enzymes identified thereof, and applications thereof.

### CROSS REFERENCE TO RELATED APPLICATIONS

This application claims priority to U.S. Provisional Application Ser. No. 63/422,767, filed Nov. 4, 2022, the disclosure of which is incorporated herein by reference in its entirety.

### STATEMENT OF GOVERNMENT SUPPORT

This invention was made with Government support under Grant No. 1847705, awarded by the National Science Foundation, Grant No. DE-AR0001508 awarded by the US Department of Energy, and Grant No. DP2 GM137427 awarded by the National Institutes of Health. The Government has certain rights in the invention.

### TECHNICAL FIELD

Provided herein is a growth selection platform for noncanonical cofactor oxidizing enzymes, the noncanonical cofactor oxidizing enzymes identified thereof, and applications thereof.

### BACKGROUND

Noncanonical cofactor biomimetics (NCBs) possess the reactive moieties essential for catalysis and carry several structural modifications that distinguish them from the natural cofactors nicotinamide adenine dinucleotide NAD(H) and nicotinamide adenine dinucleotide phosphate NADP(H). NCBs offer distinct advantages over NAD(P)/H such as lower production cost, expanded chemistries and, in some cases improved stability; NCBs also have the potential to minimize crosstalk between engineered pathways and native metabolic pathways.

### SUMMARY

Noncanonical cofactor biomimetics (NCBs) such as nicotinamide mononucleotide (NMN⁺) provide enhanced scalability for biomanufacturing. However, engineering enzymes to accept NCBs is difficult. The disclosure provides methods to identify and/or engineer enzymes that utilize NMN⁺-based reducing power by using self-sufficient growth selection. The methods disclosed herein are based on an orthogonal, NMN⁺-dependent glycolytic pathway in Escherichia coli which can be coupled to any reciprocal enzyme to recycle the ensuing reduced NMN¹ (NMNH). With a throughput of >10⁶ variants per iteration, the methods of the disclosure identified a Lactobacillus pentosus NADH oxidase variant with ~10-fold increase in NMNH catalytic efficiency and enhanced activity for other NCBs. Importantly, molecular modeling and experimental validation suggest that the mutations, instead of directly contacting NCBs, optimize the enzyme's global conformational dynamics to resemble that with the native cofactor bound. Thus, restoring the enzyme's conformation states, through deep navigation of protein sequence space by high-throughput evolution, provides a universal route to engineer NCB-dependent enzymes.

In a particular embodiment, the disclosure provides a recombinant microorganism comprising: disruption of one or more genes utilized by the microorganism for glucose metabolism; a first heterologous polypeptide encoding an orthogonal noncanonical cofactor-dependent glucose dehydrogenase (GDH ortho) and a glucose facilitated porin; and a second heterologous polypeptide encoding polypeptides which promote intracellular synthesis of the noncanonical cofactor, wherein the noncanonical cofactor can be found in both a reduced and a non-reduced form, and wherein growth of the recombinant microorganism is controlled by the availability of the noncanonical cofactor. In another embodiment or further embodiment of any of the foregoing, the microorganism is a yeast, an archaeon, or a bacterium. In another embodiment or further embodiment of any of the foregoing, the bacterium is selected from Escherichia Coli, Bacillus subtilis, Bacillus brevis, Bacillus megaterium, Brevibacillus choshinensis, Caulobacter crescentus, and Lactococcus lactis. In another embodiment or further embodiment of any of the foregoing, the bacterium is Escherichia Coli. In another embodiment or further embodiment of the foregoing, the first heterologous polypeptide is expressed from a plasmid. In another embodiment or further embodiment of any of the foregoing, the second heterologous polypeptide is expressed from a plasmid. In another embodiment or further embodiment of any of the foregoing, the first heterologous polypeptide and the second heterologous polypeptide are expressed from the same plasmid. In another embodiment or further embodiment of any of the foregoing, the first heterologous polypeptide and the second heterologous polypeptide are expressed from different plasmids. In another embodiment or further embodiment of any of the foregoing, the plasmids have low to medium copy numbers in the recombinant microorganism. In another embodiment or further embodiment of any of the foregoing, the second heterologous polypeptide is integrated into a chromosome of the recombinant microorganism. In another embodiment or further embodiment of any of the foregoing, the GDH ortho comprises a sequence that is least 95% identical to the sequence of SEQ ID NO:11. In another embodiment or further embodiment of any of the foregoing, the GDH ortho comprises the sequence of SEQ ID NO:11. In another embodiment or further embodiment of any of the foregoing, the glucose facilitated porin comprises a sequence that is least 98% identical to the sequence of SEQ ID NO: 14. In another embodiment or further embodiment of any of the foregoing, the glucose facilitated porin comprises the sequence of SEQ ID NO:14. In another embodiment or further embodiment of any of the foregoing, the polypeptides that promote intracellular synthesis of the noncanonical cofactor comprise polypeptide(s) for NAD⁺ synthase and/or nicotinate phophoribosyltransferase. In another embodiment or further embodiment of any of the foregoing, the polypeptide for NAD⁺ synthase has a sequence that is at least 99% identical to the sequence of SEQ ID NO:12. In another embodiment or further embodiment of any of the foregoing, the polypeptide for NAD⁺ synthase comprises the sequence of SEQ ID NO:12. In another embodiment or further embodiment of any of the foregoing, the polypeptide for nicotinate phophoribosyltransferase has a sequence that is at least 99% identical to the sequence of SEQ ID NO: 13. In another embodiment or further embodiment of any of the foregoing, the polypeptide for nicotinate phophoribosyltransferase comprises the sequence of SEQ ID NO:13. In another embodiment or further embodiment of any of the foregoing, the one or more genes utilized by the microorganism for glucose metabolism that are disrupted comprises phosphoglucose isomerase pgi gene, 6P-gluconate dehydrogenase gnd gene, and/or glucose-6-phosphate 1-dehydrogenase zwf gene. In another embodiment or further embodiment of any of the foregoing, the phosphoglucose isomerase pgi gene encodes a phosphoglucose isomerase having the sequence of SEQ ID NO: 15; wherein the 6P-gluconate dehydrogenase gnd gene encodes a 6P-gluconate dehydrogenase having the sequence of SEQ ID NO: 17; and wherein the glucose-6-phosphate 1-dehydrogenase zwf gene encodes a glucose-6-phosphate 1-dehydrogenase having the sequence of SEQ ID NO: 16. In another embodiment or further embodiment of any of the foregoing, the microorganism further comprises disruption of one of more genes to minimize noncanonical cofactor degradation. In another embodiment or further embodiment of any of the foregoing, the one of more genes to minimize noncanonical cofactor degradation comprises pncC gene, and/or nadR gene. In another embodiment or further embodiment of any of the foregoing, the pmcC gene encodes a nicotinamide-nucleotide aminohydrolase having the sequence of SEQ ID NO:18; and wherein the nadR gene encodes a nicotinamide-nucleotide adenylyltransferase having the sequence of SEQ ID NO: 19. In another embodiment or further embodiment of any of the foregoing, the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl) 1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. In another embodiment or further embodiment of any of the foregoing, the noncanonical cofactor is NMN. In another embodiment or further embodiment of any of the foregoing, the recombinant microorganism further comprises a third heterologous polypeptide that encodes a recombinantly engineered polypeptide that utilizes the reduced form of the noncanonical cofactor; and wherein expression of the recombinantly engineered polypeptide enhances the growth of the recombinant microorganism. In another embodiment or further embodiment of any of the foregoing, the growth of the recombinant microorganism is influenced by the specificity of the recombinantly engineered polypeptide for the reduced form of the noncanonical cofactor, wherein the higher the specificity of the recombinantly engineered polypeptide for the reduced form of the noncanonical cofactor correlates with a faster growth rate of the recombinant microorganism. In another embodiment or further embodiment of any of the foregoing, the third heterologous polypeptide is expressed from a plasmid. In another embodiment or further embodiment of any of the foregoing, the plasmid has high copy numbers in the recombinant microorganism. In another embodiment or further embodiment of any of the foregoing, the recombinantly engineered polypeptide has oxidoreductase activity.

In a certain embodiment, the disclosure also provides a growth selection process to screen recombinantly engineered polypeptides that have activity for the reduced form of a noncanonical cofactor, the growth selection process comprising: culturing a recombinant microorganism disclosed herein using a first selection criteria to identify recombinantly engineered polypeptides that have activity for the reduced form of a noncanonical cofactor by identifying recombinant microorganisms that exhibit increased growth and/or growth rate; optionally, culturing the recombinant microorganism using additional selection criteria, wherein the additional selection criteria make it more, and more, challenging for the recombinant microorganisms to grow without having activity for the reduced form of a noncanonical cofactor. In another embodiment or further embodiment of any of the foregoing, a plurality of recombinantly engineered polypeptides is screened using the growth selection process, wherein each of the plurality recombinantly engineered polypeptides vary from each other by having a different polypeptide sequence. In another embodiment or further embodiment of any of the foregoing, the polypeptide sequences for each of the recombinantly engineered polypeptides vary by length and/or by composition. In another embodiment or further embodiment of any of the foregoing, the plurality of recombinantly engineered polypeptides are variants of a wild type polypeptide sequence and comprises 1, 2, 3, 4, 5, 6, 7, 8, 9, 10, 11, or 12 introduced amino acid substitutions in comparison to the wild type polypeptide sequence. In another embodiment or further embodiment of any of the foregoing, the growth selection process is carried out using agar plates and identifying colonies which grow faster and/or larger than other colonies indicating that the recombinantly engineered polynucleotides have greater efficiencies for the reduced form of the noncanonical cofactor. In another embodiment or further embodiment of any of the foregoing, the recombinantly engineered polypeptides have oxidoreductase or oxidase activity. In another embodiment or further embodiment of any of the foregoing, the recombinantly engineered polypeptides have sequence that is at least 98% identical to a polypeptide sequence of an oxidoreductase or an oxidase from a microorganism. In another embodiment or further embodiment of any of the foregoing, the oxidoreductase or the oxidase is selected from NADH Oxidase, NADPH Oxidase, and XenA.

In a certain embodiment, the disclosure further provides a recombinantly engineered polypeptide that has a sequence that is at least 98% identical to a sequence of SEQ ID: NO 2, 3, 4, 5, 6, 7, 8, 9 or 10 and which has activity for the reduced form of a noncanonical cofactor but has limited to no activity for the natural cofactor. In another embodiment or further embodiment of any of the foregoing, the recombinantly engineered polypeptide comprises the sequence of SEQ ID NO:4, 5, 7 or 10.

In a certain embodiment, the disclosure provides for an isolated polypeptide that encodes a recombinantly engineered polypeptide disclosed herein.

In a particular embodiment, the disclosure also provides a vector or plasmid which comprises an isolated polypeptide disclosed herein.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1A-E demonstrates NMN⁺ regeneration drives dynamic glucose-dependent cell growth. (A) Engineered E. coli ED pathway couples cycling of NMN(H) to alternative glucose metabolism required for growth. Modified NMN⁺ pathways enhance intracellular NMN⁺ accumulation. Expression of NAD⁺ synthase (Ft NadE) and nicotinate phophoribosyltransferase (Ft NadV) with nicotinamide supplementation increases NMN⁺ availability, while deletion of pncC and nadR minimizes NMN⁺ degradation. (B) Reconfiguration of expression systems between strain MX502 and MX503. Strain MX502 requires three plasmids to support growth. In strain MX503, the Ft NadEV operon was integrated into the genome. (C) Specific activities of redox partners toward NMNH. From left to right redox partners were NADH Oxidase (Lb Nox), NADPH Oxidase (TP Nox), WT XenA (Broad specificity), XenA D116E (Broad specificity). For XenA WT and XenA D116E, n=3 biologically independent replicates. For all other samples, n=2 biologically independent replicates. For statistics, XenAWT to XenA D116E, p=0.00054. (D) Validation of selection pressure in MX502. Four different redox partners were expressed in MX502 and strains with 20 g/L glucose as the sole carbon source. Growth of strains was dependent on NMNH activity of assayed redox partners and the concentration of NMN⁺ supplemented in the media. (E) Validation of selection pressure in MX503. Validation reconfigured selection strain MX503 is capable of glucose-dependent growth without exogenous NMN⁺ addition. The limited NMN⁺ availability provided a more stringent selection condition compared to MX502 in D, and amplified growth differences between the XenA variants. Values are the average of two or three biological replicates with error bars representing one standard deviation. The statistical significance was determined by two-tail t-tests, *=P<0.05.

FIG. 2A-C shows redox partners' specific enzymatic activities towards NMNH, NADH and NADPH at a concentration of 0.3 mM. (A) Specific activities of an NADH-specific oxidase from L. brevis (Lb Nox) towards different cofactors. Lb Nox only has measurable activity towards its native cofactor, NADH. For all samples, n=2 biologically independent replicates. (B) Specific activities of an NADPH-specific oxidase (Triphosphopyridine nucleotide oxidase, TP Nox) towards different cofactors. Like Lb Nox, TP Nox has much higher activity towards native cofactor, NADPH, than the other two. For all samples, n=2 biologically independent replicates. (C) Broad cofactor specificity of XenA, an enoate reductase from P. putida. XenA WT has broad cofactor specificity including NMNH compared to Lb Nox and TP Nox. The mutant, XenA D116E, exhibited even higher activity towards all cofactors compared to XenA WT. For NMNH-dependent activity assay, n=3 biologically independent replicates. For all other samples, n=2 biologically independent replicates. For statistics, XenA WT to XenA D116E, p=0.00054. Data are presented as mean±standard deviation. Error bars are not displayed for sample size n=2 assay. The statistical significance was determined by two-tail t-tests (**: p<0.005).

FIG. 3 shows a robust growth phenotype of densely plated cells suggesting efficient glucose consumption. Selection strain MX502 expressing either Lb NOX (NADH oxidase), TP NOX (NADPH oxidase), XenA WT (broad cofactor specificity), or XenA D116E (increased activity compared to WT and broad cofactor specificity) was plated on selection media supplemented with 0, or 2 mM NMN⁺ and was incubated at 30 °C for an extended growth period (14 days) to heighten contrast of growth between strains expressing NMNH active or inactive redox partners. Supplementation of NMN⁺ in media can support minimal conversion of glucose to gluconate. Isolated colonies likely have extensive access to the NMN¹ present in the media to enable growth, while clustered cells are highly limited and required active NMN(H) cycling to support continued growth. However, an active redox partner is essential to efficient and sustained glucose utilization. Although growth rate of single colonies was indicative of NMNH cycling activity, robust clustered growth after re-streaking onto fresh selection media was more predictive of true positives from selection.

FIG. 4 demonstrates the growth rate of selection strains compared to wild-type E. coli strain. Growth of wild type (BW25113), MX 501, MX502 and MX503 harboring GDH Ortho and XenA was monitored in M9 minimal media with 20 g/L D-glucose and 0, 2, or 5 mM NMN⁺ supplementation. All strains possess the gene for initial glucose entry (a glucose facilitated diffusion porin from Zymomonas mobilis (Zm Glf). Selection strains MX503 and MX502 show growth dependence on extracellular NMN⁺ supplement while BW 25113 does not. Without heterologous expression of the NMN⁺ producing gene (Francisella tularensis, NadEV) to help built up intracellular NMN⁺ pool, MX 501 is not able to grow, suggesting that in vivo NMN⁺ biosynthesis still plays an essential role in selection strain growth, even when exogenous NMN⁺ is supplied. Notably, while the selection strains exhibited sustained growth and approached similar final cell density to that of wild type, the growth rates were still slow. This may originate from the disruption of the heavily utilized EMP glycolysis, which cannot be fully replaced by the ED pathway, which naturally sustains a much lower carbon flux due to the regulatory control of E. Coli. For MX501 with 0 or 2 mM NMN⁺ supplementation, MX503 with 0, 2, or 5 mM NMN⁺ supplementation, n=2 biologically independent replicates. For all other samples, n=3 biologically independent replicates.

FIG. 5A-B presents rational design of Lactobacillus pentosus NADH oxidase (Lp Nox) to utilize NMNH. (A) Model of Lp NOX positions selected for first round rational design highlighted as purple spheres in Lp NOX homology model with NADH and FAD bound. D177 is found on the end of the second Rossman beta strand and is known to regulate specificity between NADPH and NADH. Due to its active role discriminating against NADPH, we expect that it may affect NMNH selectivity as well. G154 and G241 are on different loops that compose the adenine cleft, mutations at these positions would alter the volume of the binding pocket and create steric hindrance preventing the larger NADH from fitting, binding of the smaller NMNH is expected to be minimally affected. G156, Y157, 1158, and G159 are found at the N-terminal region of the first Rossman alpha helix, these residues align the helix against the NADH pyrophosphate and create a set of polar backbone contacts and positive charge dipole to latch onto the cofactor. Substitutions in this region would reposition the helix to allow more favorable interactions with the NMNH phosphate. P295 is located across the NMINH ribose hydroxyls and mutation may create novel polar interaction. (B) Specific enzymatic activities of the rational design mutants towards 0.3 mM NMNH. Compared to wild type, 1158S and D177N exhibit ~2-fold or ~3-fold increased NMNH activity, respectively. While the mechanism of 1158S can be readily rationalized as creating a novel hydrogen bond with the phosphate of NMNH, D177N may be playing a global and indirect role based on its far distance from NMNH. Therefore, D177 is determined as an ideal site for subsequent semi-rational engineering, where I158S is fixed. Compared to the data in FIG. 6A, these data were generated using a slightly different method where 0.15 mM FAD supplementation in protein purification was omitted. Values represent the mean the two biological replicates. For statistics, WT to 1158S, p=0.0011; WT to D177N, p=0.00041. The statistical significance was determined by two-tail t-tests (**: p<0.005).

FIG. 6A-F presents Lp Nox variants discovered through growth selection. (A) Enzyme specific activities with NMNH. Wild-type Lp Nox showed an initial level of activity that was increased ~2-fold with rational design of 1158S. Growth selection of combinatorial mutants generated through the first round and second round of site-saturation mutagenesis at the cofactor binding site and the second sphere, respectively, led to LP 3, LP 7, and LP 7-1, LP 7-2. A final step of random mutagenesis with error-prone PCR led to LP 3-EP that displays ~10-fold increase in NMNH activity compared to the wild type. The dashed line under the x-axis indicates the parental variants and different selection strategies. For all samples, n=3 biologically independent replicates. For statistics, WT to I158S, p=0.00038; I158S to LP 3, p=0.012; 1158S to LP 7, p=0.0023; LP 7 to LP 7-1, p=0.0016; 1158S to LP 7-2, p=0.045; LP 3 to LP 3-EP, p=0.0014. (B) Model of LP 7 binding pose with FAD and NMNH. 1158S forms a polar contact with the NMNH phosphate, while D177W and V240L rigidly fill the binding cleft where the adenosine would typically occupy. (C) LP 3 carries an additional G178E compared to LP 7. D177W must rotate further toward the inside of the pocket to accommodate the bulkier G178E. (D, E) LP 7-1, and LP 7-2 carry second-sphere mutations that rigidify the flexible regions on the protein surface through denser packing and new polar interactions, which enhances the protein stability. (F) Superposition of Lp Nox homology model with quaternary assembly of Lb Nox (PDB: 5VN0). Mutation positions lining the cofactor pocket are colored violet. The substitutions identified from random mutagenesis P362H and V395L are remote from the active site and have an indirect effect on cofactor binding. Their surface exposure may affect multimeric assembly in addition to altering function of each individual monomer. Data are presented as mean±standard deviation. The statistical significance was determined by two tail t-tests (*: p<0.05).

FIG. 7A-B presents models of Bs GDH Triple homology with Lp Nox library selected site. (A) Lp NOX library positions highlighted as spheres in Lp NOX homology model with NADH bound. The positions were selected to be akin to those discovered to enable NMN⁺ activity in Bs GDH Triple. D177 and G178 are located similarly to Y39 on the specificity loop in GDH, and V240 resembles A93 in packing against the end of the adenosine cleft. (B) Bs GDH Triple homology model with mutated positions that correspond to Lp NOX highlighted in orange spheres. Y39 and A93 (sphere) enclose the adenosine ring.

FIG. 8 presents a logo plot summarizing the frequency of mutations at each site resulting from library selection. Strong convergence on hydrophobic and bulky residues was observed at positions 177 and 240. Individual variant sequences are available in Table 4.

FIG. 9 demonstrates the cooperative effect of Lp Nox variants mutations surrounding the adenosine ring. Compared to the wild-type Lp Nox (WT), the single mutation 1158S increased NMNH-dependent activity. However, adding V240L to 1158S did not further increase the activity significantly, and adding D177W to 1158S resulted in very poor expression possibly due to impaired protein folding. Importantly, in LP 7 (1158S-D177W-V240L) where both V240L and D177W were combined with 1158S, the activity increased ~2-fold compared to 1158S, and the expression level was restored. These results are consistent with a non-additive, cooperative effect between V240L and D177W, which are located at the opposing sides of the adenosine binding cleft and predicted to pack against each other via hydrophobic interaction. The models indicate that the dense packing mitigates Lp Nox's excessive flexibility when utilizing the much smaller non-canonical cofactors compared to NADH and promotes robust cofactor binding and catalysis. V240L alone does not afford the activity enhancing effect. The lone hydrophobic mutation D177W on the protein surface may cause a thermodynamic penalty associated with the extra energy needed for its solvation, resulting in poor folding. This penalty is compensated by the addition of V240L which forms stabilizing interaction with D177W and makes it more deeply embedded. Specific enzymatic activity was measured with NMNH at 0.3 mM. For all samples, n=3 biologically independent replicates. For statistics, 1158S to 1158S-V240L, p=0.322, not significant; I158S to LP-7, p=0.0023; I158S-V240L to LP-7, p=0.0017. Data are presented as mean±standard deviation. The statistical significance was determined by two-tail t-tests (**: p<0.005).

FIG. 10A-B demonstrates that variants selected from the second round of growth selection display increased solubility and thermostability. (A) SDS-PAGE analysis of protein solubility. The insoluble pellet (P) and supernatant(S) fractions obtained through centrifugation following cell lysis are evaluated for Nox expression. Variant LP 7-1 and LP 7-2 show increased solubility fraction (lane 10/11 and 12/13) compared to LP 7 (lane 8/9). WT Lp Nox (lane 4/5) and empty plasmid harboring samples (lane 1/2) serve as controls. (B) Relative residual activity before and after heat treatment. After 23 h of heat treatment at 50 °C, WT activity with native cofactor was diminished to 7%+2%. Meanwhile engineered variants LP 7, LP 7-1, and LP 7-2 show enhanced thermal stability. Moreover, LP 7-2 (30%±6%) displays significantly higher thermal stability than LP 7 (19%±3%). Data are presented as the ratio between the average of three biologically independent replicates±error. Scattered data points represent the ratio between individual samples after heat treatment and the averaged measurement before treatment. Error in relative residual activity is calculated by propagation of the error in treated and untreated samples' measurement.

FIG. 11A-B provides enzymatic specific activities of engineered Lp Nox variants towards other biomimetic cofactors. (A) Compared to WT Lp Nox, engineered Lp Nox variant (LP3-EP) displayed ~2.5-fold higher activity towards MNAH. Without the I158S mutation to form the hydrogen bond with the phosphate, LP3-EP 1158 still shows similar MNAH activity suggesting the remaining mutations function allosterically since they should not have direct contact with the binding pose of MNAH. For LP 3-EP, n=4 biologically independent replicates. For other samples, n=3 biologically independent replicates. For statistics, WT to LP 3-EP, p=0.000074; WT to LP 3-EP 1158, p=0.00097. (B) Similar to the activity trend with MNAH, Lp Nox variant (LP-3-EP) shows a ~4-fold increase in specific activity compared to WT Lp Nox using BNAH. Furthermore, LP-3-EP 1158 still shows higher activity with BNAH than WT Lp Nox. For all samples, n=4 biologically independent replicates. For statistics, WT to LP 3-EP, p=2.5×10−7; WT to LP 3-EP 1158, p=0.0014. Data are presented as mean±standard deviation. The statistical significance was determined by two-tail t-tests, (*: p<0.05).

FIG. 12A-C provides a comparison of Rossman fold loop flexibility between wild type and engineered Lp Nox. 40 random frames are selected and aligned from each simulation to visualize the range of conformations sampled. The loop regions measured are highlighted (residues 153-157, 177-188, and 238-242) and envelope the AMP portion of the native NADH cofactor. The three mutated positions in the binding site (D177W, G178E, and V240L) are indicated as spheres. (A) WT with MNAH bound displays high flexibility as indicated by large displacement of the strands. The fluctuations impede organization of the active site to support cofactor binding and catalysis. (B) Mut with MNAH bound shows more tightly packed bundles for the overlayed structures, demonstrating greater rigidity compared to the WT. The addition of the large, hydrophobic substitutions crowds the AMP binding cleft and reduces the volume of space for the loops to explore alternative conformations. (C) Quantification of the loop flexibility through alpha carbon RMSD illustrates that WT with MNAH exhibits higher loop flexibility with 0.94±0.13 Å in comparison to Mut with MNAH at 0.75±0.08 Å or WT with NADH at 0.74±0.10 Å.

FIG. 13A-B provides measurement of hydride transfer efficiency of wild type and engineered Lp Nox. (A) Electrons are shuttled from the nicotinamide cofactor to FAD through movement between the nicotinamide ring C4 and FAD N5. A catalytically competent enzyme must be able to support this contact, otherwise catalysis cannot occur. A threshold of 4.5 Å is bounded to categorize binding poses below as being active and poses below as being active and poses with distances above as being inactive since the cofactors are too far to engage in hydride transfer. (B) WT with MNAH bound samples the active pose with only 1% frequency. The inability to maintain the MNAH within hydride transfer distance is resolved in the Mut (LP 3-EP 1158) which samples the active pose at 60% frequency. The WT Lp NOX firmly anchors the native NADH through the AMP handle and occupies the active pose in 74% of the frames.

FIG. 14A-D presents a comparison of conformational landscape of wild type and engineered Lp Nox. Global variation in backbone ensembles is represented by PCA dimensionality reduction of alpha carbon dihedrals, all samples are combined onto shared latent space and plotted on the first two principal components. Color intensity in panels B, C, and D indicate temporality, with lighter gray signifying conformations at the start of the simulation, and darker gray marking samples near the end. (A) The combined free energy landscape shows grouping into three local minima, whose centers are labeled A, B, and C through K-Means clustering. (B) WT with MNAH samples disjoint regions of conformation space along the first principal component corresponding to clusters A and C, and rarely traverses through the central cluster B. The replicates do not overlap, suggesting a high degree of flexibility and divergence in conformations. (C) Conformations of WT with NADH are focused on cluster B which is spread more broadly along the second principal component. (D) Mut (LP 3-EP 1158) with MNAH occupies cluster B and does not stray into other regions, similar to WT with NADH. The Mut with MNAH surveys a comparable ensemble of backbone conformations that are characterized by catalytic ability to the WT with NADH.

FIG. 15 provides a polypeptide sequence (SEQ ID NO:1) for a wild type Lactobacillus pentosus H₂O-forming NADH oxidoreductase.

FIG. 16 provides a polypeptide sequence (SEQ ID NO:2) for a variant Lactobacillus pentosus H₂O-forming NADH oxidase, wherein the sequence comprises a 1158S substitution in comparison to the sequence of FIG. 15.

FIG. 17 provides a polypeptide sequence (SEQ ID NO:3) for a variant Lactobacillus pentosus H₂O-forming NADH oxidase, wherein the sequence comprises I158S, D177W, and V240L substitutions (LP 7) in comparison to the sequence of FIG. 15.

FIG. 18 provides a polypeptide sequence (SEQ ID NO:4) for a variant Lactobacillus pentosus H₂O-forming NADH oxidase, wherein the sequence comprises 1158S, D177W, L179H, Q210C, M211W and V240L substitutions (LP 7-1) in comparison to the sequence of FIG. 15.

FIG. 19 provides a polypeptide sequence (SEQ ID NO:5) for a variant Lactobacillus pentosus H₂O-forming NADH oxidase, wherein the sequence comprises I158S, D177W, L179E, Q210V, M211S, and V240L substitutions (LP 7-2) in comparison to the sequence of FIG. 15.

FIG. 20 provides a polypeptide sequence (SEQ ID NO:6) for a variant Lactobacillus pentosus H₂O-forming NADH oxidase, wherein the sequence comprises I158S, D177W, G178E, and V240L substitutions (LP 3) in comparison to the sequence of FIG. 15.

FIG. 21 provides a polypeptide sequence (SEQ ID NO:7) for a variant Lactobacillus pentosus H₂O-forming NADH oxidase, wherein the sequence comprises I158S, D177W, G178E, V240L, P362H and V395L substitutions (LP 3-EP) in comparison to the sequence of FIG. 15.

FIG. 22 provides a polypeptide sequence (SEQ ID NO:8) for a variant Lactobacillus pentosus H₂O-forming NADH oxidase, wherein the sequence comprises D177W and V240L substitutions in comparison to the sequence of FIG. 15.

FIG. 23 provides a polypeptide sequence (SEQ ID NO:9) for a variant Lactobacillus pentosus H₂O-forming NADH oxidase, wherein the sequence comprises D177W, G178E and V240L substitutions in comparison to the sequence of FIG. 15.

FIG. 24 provides a polypeptide sequence (SEQ ID NO:10) for a variant Lactobacillus pentosus H₂O-forming NADH oxidase, wherein the sequence comprises D177W, G178E, V240L, P362H and V395L substitutions (LP 3-EP 1158) in comparison to the sequence of FIG. 15.

FIG. 25 provides a polypeptide sequence (SEQ ID NO:11) for an orthogonal noncanonical cofactor-dependent glucose dehydrogenase (GDH ortho).

FIG. 26 provides a polypeptide sequence (SEQ ID NO:12) for Francisella tularensis subsp. tularensis NH₃-dependent NAD⁺ synthase (Ft NadE). Uniprot ID: Q5NFH5.

FIG. 27 provides a polypeptide sequence (SEQ ID NO:13) for Francisella tularensis subsp. tularensis nicotinate phosphoribosyltransferase (Ft NadV). Uniprot ID: A0A0E3A662.

FIG. 28 provides a polypeptide sequence (SEQ ID NO:14) for Zymomonas mobilis glucose facilitated diffusion porin (Zm Glf). Uniprot ID: P21906.

FIG. 29 provides a polypeptide sequence (SEQ ID NO:15) for Escherichia coli glucose-6-phosphate isomerase (Ec Pgi). Uniprot ID: P0A6T1.

FIG. 30 provides a polypeptide sequence (SEQ ID NO:16) for Escherichia coli glucose-6-phosphate 1-dehydrogenase (Ec Zwf). Uniprot ID: P0AC53.

FIG. 31 provides a polypeptide sequence (SEQ ID NO:17) for Escherichia coli 6-phosphogluconate dehydrogenase (Ec Gnd). Uniprot ID: P00350.

FIG. 32 provides a polypeptide sequence (SEQ ID NO:18) for Escherichia coli nicotinamide-nucleotide aminohydrolase (Ec PncC). Uniprot ID: P0A6G3.

FIG. 33 provides a polypeptide sequence (SEQ ID NO:19) for Escherichia coli nicotinamide-nucleotide adenylyltransferase (Ec NadR). Uniprot ID: P27278.

FIG. 34 provides a polypeptide sequence (SEQ ID NO:20) for Pseudomonas putida NADH: flavin oxidoreductase/NADH oxidase (Pp XenA). Uniprot ID: Q9R9V9.

FIG. 35 provides a polypeptide sequence (SEQ ID NO:21) for Levilactobacillus brevis FAD-dependent oxidoreductase (Lb Nox). PDB: 5VN0.

FIG. 36 provides a polypeptide sequence (SEQ ID NO:22) for a variant of Levilactobacillus brevis FAD-dependent oxidoreductase (TP Nox). PDB: 5VOH.

### DETAILED DESCRIPTION

As used herein and in the appended claims, the singular forms “a”, “an”, and “the” include plural referents unless the context clearly dictates otherwise. Thus, for example, reference to “a polynucleotide” includes a plurality of such polynucleotides and reference to “the enzyme” includes reference to one or more enzymes, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise”, “comprises”, “comprising”, “include”, “includes”, and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated by reference in full for the purpose of describing and disclosing methodologies that might be used in connection with the description herein. Moreover, with respect to any term that is presented in one or more publications that is similar to, or identical with, a term that has been expressly defined in this disclosure, the definition of the term as expressly provided in this disclosure will control in all respects.

As used herein, a “natural cofactor” or “canonical cofactor” refers to a non-protein chemical compound or metallic ion that is normally required for an enzyme's activity as a catalyst. Natural cofactors can be either loosely or tightly bound to the enzyme and can directly participate in the reaction. For example, NAD⁺ or NADP⁺ are natural cofactors for glucose dehydrogenase that are required for the enzyme's activity. In direct contrast, NMN¹ is not a natural cofactor for glucose dehydrogenase, and therefore is not required for the enzyme's in vivo activity.

As used herein, a “non-natural cofactor” or “noncanonical cofactor” refers to a chemical compound or metallic ion that is not normally required or associated with a particular enzyme's activity as a catalyst, but by the result of mutations or other changes, the enzyme's activity towards the noncanonical cofactor can be greatly enhanced. For example, NMN⁺ is not a natural cofactor for glucose dehydrogenase, but by introducing mutations into the polypeptide sequence for glucose dehydrogenase, the glucose dehydrogenase's activity towards NMN⁺ can be greatly enhanced.

As used herein, a “mutation” means any process or mechanism resulting in a mutant protein, enzyme, polynucleotide, gene, or cell. This includes any mutation in which a protein, enzyme, polynucleotide, or gene sequence is altered, and any detectable change in a cell arising from such a mutation. Typically, a mutation occurs in a polynucleotide or gene sequence, by point mutations, deletions, or insertions of single or multiple nucleotide residues. A mutation includes polynucleotide alterations arising within a protein-encoding region of a gene as well as alterations in regions outside of a protein-encoding sequence, such as, but not limited to, regulatory or promoter sequences. A mutation in a gene can be “silent”, i.e., not reflected in an amino acid alteration upon expression, leading to a “sequence-conservative” variant of the gene. This generally arises when one amino acid corresponds to more than one codon. In certain embodiments, a mutation in a gene result in the change of an amino acid in an expressed polypeptide sequence. For purposes of this disclosure, changes like the foregoing, are termed “amino acid substitutions” and can include 1, 2, 3, 4, 5, 6, 7, 8, 9, 10, 11, 12 or more amino acid changes in an expressed polypeptide sequence.

Recombinant methods for producing and isolating modified polypeptides of the disclosure are described herein. In addition to recombinant production, the polypeptides may be produced by direct peptide synthesis using solid-phase techniques (e.g., Stewart et al. (1969) Solid-Phase Peptide Synthesis (W H Freeman Co, San Francisco); and Merrifield (1963) J. Am. Chem. Soc. 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer.

A “reductase,” as used herein, refers to an enzyme that catalyzes the transfer of electrons from one molecule, the reductant, also called the electron donor, to another, the oxidant, also called the electron acceptor. An “oxidoreductase,” as used herein, refers to an enzyme that allows an oxidation to occur as well as a reduction to occur. Examples of oxidoreductases and reductases include those enzymes that act on the CH—OH group of donors with NAD⁺ or NADP⁺ as an acceptor, including alcohol dehydrogenase (NAD) (EC 1.1.1.1), alcohol dehydrogenase (NADP) (EC 1.1.1.2), homoserine dehydrogenase (EC 1.1.1.3), aminopropanol oxidoreductase (EC 1.1.1.4), diacetyl reductase (EC 1.1.1.5), glycerol dehydrogenase (EC 1.1.1.6), propanediol-phosphate dehydrogenase (EC 1.1.1.7), glycerol-3-phosphate dehydrogenase (NAD⁺) (EC 1.1.1.8), D-xylulose reductase (EC 1.1.1.9), L-xylulose reductase (EC 1.1.1.10), lactate dehydrogenase (EC 1.1.1.27), malate dehydrogenase (EC 1.1.1.37), isocitrate dehydrogenase (EC 1.1.1.42), and HMG-COA reductase EC (1.1.1.88); enzymes that act on the CH—OH group of donors with oxygen as an acceptor, including glucose oxidase (EC 1.1.3.4), L-gulonolactone oxidase (EC 1.1.3.8), thiamine oxidase (EC 1.1.3.23), xanthine oxidase (EC 1.1.3.32); enzymes that act on the aldehyde or oxo group of donors with NAD⁺ or NADP⁺ as an acceptor, including acetaldehyde dehydrogenase EC (1.2.1.10), glyceraldehyde 3-phosphate dehydrogenase (EC 1.2.1.12), pyruvate dehydrogenase (EC 1.2.1.51), oxoglutarate dehydrogenase (EC 1.2.4.2); enzymes that act on the CH—CH group of donors with NAD⁺ or NADP⁺ as an acceptor, including biliverdin reductase (EC 1.3.1.24); enzymes that act on CH—CH group of donors with oxygen as an acceptor, including protoporphyrinogen oxidase (EC 1.3.3.4); enzymes that act on the CH—NH₂ group of donors, including monoamine oxidase (EC 1.4.3.4); enzymes that act on the CH—NH group of donors with NAD⁺ or NADP⁺ as an acceptor, including dihydrofolate reductase (EC 1.5.1.3), and methylenetetrahydrofolate reductase (EC 1.5.1.20); enzymes that act on the CH—NH group of donors with oxygen as an acceptor, sarcosine oxidase (EC 1.5.3.1), and dihydrobenzophenanthridine oxidase (EC 1.5.3.12); enzymes that act on other nitrogenous compounds as donors, including urate oxidase (EC 1.7.3.3), nitrite reductase (EC 1.7.99.3), and nitrate reductase (EC 1.7.99.4); enzymes that act on the sulfur group of donors, including glutathione reductase (EC 1.8.1.7), thioredoxin reductase (EC 1.8.1.9), and sulfite oxidase (EC 1.8.3.1); enzymes that act on the heme group of donors, including cytochrome c oxidase (EC 1.9.3.1); enzymes that act on diphenols and related substances as donors, including coenzyme Q-cytochrome c reductase (EC 1.10.2.2), catechol oxidase (EC 1.10.3.1), and laccase (EC 1.10.3.2); enzymes that act on peroxide as acceptor, including Cytochrome c peroxidase (EC 1.11.1.5), catalase (EC 1.11.1.6), myeloperoxidase (EC 1.11.1.7), thyroid peroxidase (EC 1.11.1.8), and glutathione peroxidase (EC 1.11.1.9); enzymes that act on single donors with incorporation of molecular oxygen, 4-hydroxyphenylpyruvate dioxygenase (EC 1.13.11.27), renilla-luciferin 2-monooxygenase (EC 1.13.12.5), cypridina-luciferin 2-monooxygenase (EC 1.13.12.6), Firefly luciferase (EC 1.13.12.7), watasenia-luciferin 2-monooxygenase (EC 1.13.12.8), and oplophorus-luciferin 2-monooxygenase EC (1.13.12.13); enzymes that act on paired donors with incorporation of molecular oxygen, including aromatase (EC 1.14.14.1), CYP2D6 (EC 1.14.14.1), CYP2E1 (EC 1.14.14.1), CYP3A4 (EC 1.14.14.1), Cytochrome P450 oxidase, nitric oxide synthase (EC 1.14.13.39), phenylalanine hydroxylase (EC 1.14.16.1), and tyrosinase (EC 1.14.18.1); and other oxidoreductases, including superoxide dismutase (EC 1.15.1.1), nitrogenase (EC 1.18.6.1), and deiodinase (EC 1.97.1.10). The above listing, provides for the classification of the foregoing enzymes by in the International Union of Biochemistry and Molecular Biology's Enzyme Commission [EC] numbering system. In a particular embodiment, the disclosure provides for an engineered polypeptide based upon an oxidase or oxidoreductase disclosed herein, that has been engineered to contain amino acid mutations to enable the efficient recycling of a noncanonical cofactor. In a further embodiment, the oxidase or oxidoreductase is a NADH Oxidase (e.g., a NADH-specific oxidase from L. brevis (Lb Nox)), a NADPH Oxidase (e.g., a triphosphopyridine nucleotide oxidase (TP Nox)) and an enoate reductase (e.g., an enoate reductase from P. putida (XenA)).

“Dehydrogenase,” as used herein, refers to an enzyme belonging to the group of oxidoreductases that oxidizes a substrate by reducing an electron acceptor, usually NAD⁺/NADP⁺ or a flavin coenzyme such as FAD or FMN. They also catalyze the reverse reaction, for instance alcohol dehydrogenase not only oxidizes ethanol to acetaldehyde in animals but also produces ethanol from acetaldehyde in yeast. In another embodiment, the disclosure provides for an engineered polypeptide based upon a dehydrogenase that has been engineered to contain amino acid mutations so as to enable the efficient recycling of a noncanonical cofactor.

A “protein” or “polypeptide”, which terms are used interchangeably herein, refers to one or more chains of chemical building blocks called amino acids that are linked together by chemical bonds called peptide bonds. An “enzyme,” as used herein, refers to any substance, preferably composed wholly or largely of a protein, that catalyzes or promotes, more or less specifically, one or more chemical or biochemical reactions. A “native” or “wild-type” protein, enzyme, polynucleotide, gene, or cell refers to a protein, enzyme, polynucleotide, gene, or cell, respectively, which occurs in nature.

An “amino acid sequence” is a polymer of amino acids (a protein, polypeptide, etc.) or a character string representing an amino acid polymer, depending on context. The terms “protein” and “polypeptide” are used interchangeably herein. “Amino acid” is a molecule having the structure wherein a central carbon atom is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

As used herein, “conservative amino acid substitution” or, simply, “conservative substitution” of a particular sequence refers to the replacement of one amino acid, or series of amino acids, with different amino acids that have similar biochemical properties (e.g., charge, hydrophobicity and size). One of skill will recognize that individual mutations, deletions or additions which alter, add or delete a single amino acid or a percentage of amino acids in an encoded sequence result in “conservative variations” where the alterations result in the deletion of an amino acid, addition of an amino acid, or substitution of an amino acid with a chemically similar amino acid. For purposes of this disclosure a “conservative amino acid substitution” does significantly affect the catalytic activity towards a noncanonical cofactor and/or structural stability of an engineered polypeptide disclosed herein. For example, the engineered polypeptide of the disclosure may comprise conservative amino acid mutations in regions of the sequence that do not impact the binding site for the noncanonical cofactor, e.g., conservative amino acid changes on the surface of the protein. Further, the sequence of an engineered polypeptide disclosed herein can be aligned with polypeptide sequence(s) from enzymes that have similar structures and/or catalytic activity in order to identify amino acids that likely do not affect the catalytic activity and/or structural stability of the engineered polypeptide. Moreover, there are many protein modeling programs available, including those specifically recited herein (e.g., Spartan and RosettaDesign), which can identify conservative amino acid mutations with a high degree of probability/certainty that would not significantly affect the catalytic activity and/or structural stability of an engineered polypeptide disclosed herein (e.g., see Ng et al., Predicting Deleterious Amino Acid Changes Genome Res 11:863-874 (2001)). As such, it is expected that one of skill in the art could reasonably predict that the sequence for an engineered polypeptide disclosed herein can comprise a percentage of conservative amino acid mutations, as is described more fully below, and still have similar or the same catalytic activity for the noncanonical cofactor as a polypeptide sequence specifically recited herein (e.g., SEQ ID NO:6 or SEQ ID NO:8). Similar reasoning applies for the structural stability of an engineered polypeptide disclosed herein.

Conservative substitution tables providing functionally similar amino acids are well known in the art. For example, one conservative substitution group includes Alanine (A), Serine(S), and Threonine (T). Another conservative substitution group includes Aspartic acid (D) and Glutamic acid (E). Another conservative substitution group includes Asparagine (N) and Glutamine (Q). Yet another conservative substitution group includes Arginine (R) and Lysine (K). Another conservative substitution group includes Isoleucine, (I) Leucine (L), Methionine (M), and Valine (V). Another conservative substitution group includes Phenylalanine (F), Tyrosine (Y), and Tryptophan (W).

Thus, “conservative amino acid mutations” of a polypeptide sequence disclosed herein include mutations of a percentage, typically less than 5%, 6%, 7%, 8%, 9%, or 10%, of the amino acids of the polypeptide sequence, with a conservatively selected amino acid of the same conservative substitution group. Accordingly, a conservatively substituted variation of a polypeptide of the disclosure can contain 1, 2, 3, 4, 5, 6, 7, 8, 9, 10, 15, 20, 25, or any range that includes or is in between mutations with a conservatively substituted variation of the same conservative substitution group.

It is understood that the addition of sequences which do not alter the encoded activity of a nucleic acid molecule, such as the addition of a non-functional or non-coding sequence, is a conservative variation of the basic nucleic acid. The “activity” of an enzyme is a measure of its ability to catalyze a reaction, i.e., to “function”, and may be expressed as the rate at which the product of the reaction is produced. For example, enzyme activity can be represented as the amount of product produced per unit of time or per unit of enzyme (e.g., catalytic efficiency), or in terms of affinity or dissociation constants.

One of skill in the art will appreciate that many conservative variations of the nucleic acid constructs which are disclosed yield a functionally identical construct. For example, as discussed above, owing to the degeneracy of the genetic code, “silent mutations” (i.e., mutations in a nucleic acid sequence which do not result in an alteration in an encoded polypeptide) are an implied feature of every nucleic acid sequence which encodes an amino acid. Similarly, “conservative amino acid mutations,” in one or a few amino acids in an amino acid sequence are substituted with different amino acids with highly similar properties, are also readily identified as being highly similar to a disclosed construct. Such conservative variations of each disclosed sequence are a feature of the polypeptides provided herein.

As used herein, “conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Amino acids other than those indicated as conserved may differ in a protein or enzyme so that the percent protein or amino acid sequence similarity (or identity) between any two proteins of similar function may vary and can be, for example, at least 30%, at least 50%, at least 70%, at least 80%, or at least 90%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA (Lipman and Pearson, 1985; Pearson and Lipman, 1988). When using all of these programs, the preferred settings are those that results in the highest sequence similarity.

Non-conservative modifications of a particular polypeptide are those which substitute any amino acid not characterized as a conservative substitution. For example, any substitution which crosses the bounds of the six groups set forth above. These include mutations of basic or acidic amino acids for neutral amino acids, (e.g., Asp, Glu, Asn, or Gln for Val, Ile, Leu or Met), aromatic amino acid for basic or acidic amino acids (e.g., Phe, Tyr or Trp for Asp, Asn, Glu or Gln) or any other substitution not replacing an amino acid with a like amino acid. Basic side chains include lysine (K), arginine (R), histidine(H); acidic side chains include aspartic acid (D), glutamic acid (E); uncharged polar side chains include glycine (G), asparagine (N), glutamine (Q), serine(S), threonine (T), tyrosine (Y), cysteine (C); nonpolar side chains include alanine (A), valine (V), leucine (L), isoleucine (I), proline (P), phenylalanine (F), methionine (M), tryptophan (W); beta-branched side chains include threonine (T), valine (V), isoleucine (I); aromatic side chains include tyrosine (Y), phenylalanine (F), tryptophan (W), and histidine(H).

A “parent” protein, enzyme, polynucleotide, gene, or cell is any protein, enzyme, polynucleotide, gene, or cell, from which any other protein, enzyme, polynucleotide, gene, or cell, is derived or made, using any methods, tools or techniques, and whether or not the parent is itself native or mutant, respectively. A parent polynucleotide or gene encodes for a parent protein or enzyme. In a certain embodiment, a “parent” protein, enzyme, polynucleotide, gene, or cell, is a wild type protein, enzyme, polynucleotide, gene, or cell.

As used herein, “Isolated polypeptide” refers to a polypeptide which is separated from other contaminants that naturally accompany it, e.g., protein, lipids, and polynucleotides. The term embraces polypeptides which have been removed or purified from their naturally-occurring environment or expression system (e.g., host cell or in vitro synthesis).

As used herein, “substantially pure polypeptide” refers to a composition in which the polypeptide species is the predominant species present (i.e., on a molar or weight basis it is more abundant than any other individual macromolecular species in the composition) and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

As used herein, “reference sequence” refers to a defined sequence used as a basis for a sequence comparison. A reference sequence may be a subset of a larger sequence, for example, a segment of a full-length gene or polypeptide sequence. Generally, a reference sequence can be at least 20 nucleotide or amino acid residues in length, at least 25 nucleotide or residues in length, at least 50 nucleotides or residues in length, or the full length of the nucleic acid or polypeptide. Since two polynucleotides or polypeptides may each comprise a sequence (i.e., a portion of the complete sequence) that is similar between the two sequences and may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity.

As used herein, “sequence identity” means that two polypeptide sequences are substantially identical (i.e., on an amino acid-by-amino acid basis) over a window of comparison. The term “sequence similarity” refers to similar amino acids that share the same biophysical characteristics. The term “percentage of sequence identity” or “percentage of sequence similarity” is calculated by comparing two optimally aligned sequences over the window of comparison, determining the number of positions at which the identical residues (or similar residues) occur in both polypeptide sequences to yield the number of matched positions, dividing the number of matched positions by the total number of positions in the window of comparison (i.e., the window size), and multiplying the result by 100 to yield the percentage of sequence identity (or percentage of sequence similarity). Regarding polynucleotide sequences, the terms sequence identity and sequence similarity have comparable meaning as described for protein sequences, with the term “percentage of sequence identity” indicating that two polynucleotide sequences are identical (on a nucleotide-by-nucleotide basis) over a window of comparison. As such, a percentage of polynucleotide sequence identity (or percentage of polynucleotide sequence similarity, e.g., for silent mutations or other mutations, based upon the analysis algorithm) also can be calculated. Maximum correspondence can be determined by using one of the sequence algorithms described herein (or other algorithms available to those of ordinary skill in the art) or by visual inspection. In a particular embodiment, a polypeptide sequence will have about 80%, about 85% or more, about 90% or more, about 95% or more, and about 98% or more, sequence identity to another polypeptide sequence.

As applied to polypeptides, the term substantial identity or substantial similarity means that two peptide sequences, when optimally aligned, such as by the programs BLAST, GAP or BESTFIT using default gap weights or by visual inspection, share sequence identity or sequence similarity. Similarly, as applied in the context of two nucleic acids, the term substantial identity or substantial similarity means that the two nucleic acid sequences, when optimally aligned, such as by the programs BLAST, GAP or BESTFIT using default gap weights (described elsewhere herein) or by visual inspection, share sequence identity or sequence similarity.

One example of an algorithm that is suitable for determining percent sequence identity or sequence similarity is the FASTA algorithm, which is described in Pearson, W. R. & Lipman, D. J., (1988) Proc. Natl. Acad. Sci. USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width=16.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendrogram showing the clustering relationships used to create the alignment. PILEUP uses a simplification of the progressive alignment method of Feng & Doolittle, (1987) J. Mol. Evol. 35:351-360. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc. Acids Res. 12:387-395).

Another example of an algorithm that is suitable for multiple DNA and amino acid sequence alignments is the CLUSTALW program (Thompson, J. D. et al., (1994) Nuc. Acids Res. 22:4673-4680). CLUSTALW performs multiple pairwise comparisons between groups of sequences and assembles them into a multiple alignment based on sequence identity. Gap open and Gap extension penalties were 10 and 0.05 respectively. For amino acid alignments, the BLOSUM algorithm can be used as a protein weight matrix (Henikoff and Henikoff, (1992) Proc. Natl. Acad. Sci. USA 89:10915-10919).

Additional favorable polypeptides sequences for engineering can be identified by using sequence alignment. For example, sequences that have sequence alignment of at least 95% to a sequence of SEQ ID: 2 to SEQ ID: 9, and/or the accession numbers listed above would provide for such sequences. The modified polypeptide may then be assayed for efficiency using a noncanonical cofactor in the methods described herein.

Noncanonical cofactor biomimetics (NCBs) possess the reactive moieties essential for catalysis and carry several structural modifications that distinguish them from natural cofactors (e.g., nicotinamide adenine dinucleotide NAD(H) and nicotinamide adenine dinucleotide phosphate NADP(H)). These NCBs offer distinct advantages over NAD(P)/H such as lower production cost, expanded chemistries, and, in some cases improved stability; NCBs also have the potential to minimize crosstalk between engineered pathways and native metabolisms. Some NCBs are metabolites that naturally exist in the cells or consist of natural building blocks, which suggests their potential of being renewably biosynthesized. In particular, the noncanonical cofactor nicotinamide mononucleotide NMN(H) mimics the conventional cofactors, but with a truncated structure lacking the adenosine binding handle typically used for enzyme recognition. In E. coli whole cells and crude cell lysates, NMN⁺ can enable specific electron delivery and eliminate the need for extensive strain engineering as demonstrated by the respective 86% and 97-100% purity achieved through the NMNH-dependent whole cell and crude lysate production of the aldehyde citronellal. Nicotinamide mononucleotide (NMN⁺), a truncated version of the native nicotinamide cofactors, was found to be an efficient noncanonical redox cofactor. Compared with other simpler NAD⁺ mimetics, NMN⁺ can be produced renewably using low-cost feedstocks through biosynthetic pathways. More importantly, its structural features offer unique advantages for enzyme design: NMN⁺ retains the ribose phosphate moiety ideal for hydrogen bonding and electrostatic interactions, which enables stronger recognition than interactions based on van der Waals contacts. In biotransformation in vitro, NMN⁺ can support a diverse range of industrially important chemistries with high productivity and long-term stability. The expanding catalog of productive catalysis with NCBs create a need for rapid screening and enzyme engineering.

The concepts elucidated from the natural divergence of NADH, and NADPH specific enzymes have provided limited use in the transition to NCBs and effective design principles are needed. Previous efforts to design proteins for NCB binding has not led to clear design principles as few experimental structures with the NCB bound have been solved, and only a small fraction of the work has been devoted to understanding the mechanisms behind the improved activity in engineered variants.

Colorimetric assays have been the predominant method for screening altered cofactor preferences. Screening through use of the substrate analog p-nitrophenoxydecanoic acid can be applied in a 96-well plate format and has highlighted key residues responsible for introducing cofactor flexibility in the soluble P450 BM3. Limited throughput has motivated the development of efficient agar-based screening platforms for dehydrogenases based on colorimetric redox dyes such as tetranitro blue tetrazolium (TNBT) and water-soluble tetrazolium variants (WST). A recent application of these dyes was implemented as a NAD(P)-eliminated solid phase assay (NESPA) to screen for NMN⁺ activity 6-rounds of directed evolution to successfully identified a dehydrogenase with a 50-fold improvement of catalytic efficiency with NMN¹. While this method greatly enhanced the throughput of screening for NCB activity (10⁵ per round), the 70 °C heat-treatment required excludes applications for mesophilic enzymes. Recent expansions of the available redox-based growth selections highlight the practical use of these platforms for engineering diverse cofactor dependent enzymes with cell growth as an easy readout of activity. While these selections can increase throughput by >10⁶-fold, a link between the desired enzyme activity and a life-essential function is needed.

Reported herein is the development of a facile, high-throughput, and universal growth selection platform to screen polypeptides (e.g., recombinantly engineered polypeptides) that have activity for a noncanonical cofactor. The growth selection platform of the disclosure utilizes a highly specialized microorganism that has been recombinantly modified to disrupt the microorganism's natural glucose metabolism, and to express one or more heterologous polypeptides that encode an orthogonal noncanonical cofactor-dependent glucose dehydrogenase (GDH ortho), a glucose facilitated porin, and polypeptide(s) that promote intracellular synthesis of the nonconical cofactor. The microorganism may further comprise disruption of one of more genes to minimize noncanonical cofactor degradation (e.g., pncC gene, and/or nadR gene). In a particular embodiment, the microorganism is a bacterium, a fungus, or an archaeon. In a further embodiment, the fungus is a yeast. Examples of yeast that can be used in the methods of the disclosure include, but are not limited to, yeast from the Saccharomyces family (e.g., S. cerevisiae). Examples of bacteria that can be used in the methods disclosed herein include, but are not limited to, Escherichia Coli, Bacillus subtilis, Bacillus brevis, Bacillus megaterium, Brevibacillus choshinensis, Caulobacter crescentus, and Lactococcus lactis. In a particular embodiment, the microorganism used in the methods disclosed herein is Escherichia Coli. The heterologous polypeptide(s) may be incorporated into the genome of the microorganism, or alternatively, vectors (e.g., plasmids) may comprise the heterologous polypeptide(s). In a particular embodiment, a single heterologous polypeptide encodes GDH ortho, a glucose facilitated porin, and polypeptide(s) that promote intracellular synthesis of the nonconical cofactor. In another embodiment, a first heterologous polypeptide encodes GDH ortho and a glucose facilitated porin. In a further embodiment, a second heterologous polypeptide encodes polypeptide(s) that promote intracellular synthesis of the nonconical cofactor. In further embodiments, one or more vectors comprise the first heterologous polypeptide and/or the second heterologous polypeptide. In yet a further embodiment, the first heterologous polypeptide and/or the second heterologous polypeptide are incorporated into the genome of the microorganism. In another embodiment, the expression of the heterologous polypeptide(s) is controlled by promoters that are either constitutive or inducible. In a further embodiment, the promoters provide for low to medium level expression of the heterologous polypeptides. In yet other embodiments, the microorganisms comprise low to medium copy numbers of vectors comprising the heterologous polypeptide(s).

In regard to GDH ortho, this enzyme variant makes the life-essential reduced glutathione (GSH) using a noncanonical cofactor as the reducing equivalent specifically. As shown herein, cell growth can be coupled to noncanonical cofactor (e.g., NMN+) regeneration by wiring GDH Ortho into the central metabolism of a microorganism (e.g., E. coli). This growth phenotype has been leveraged herein to develop a high-throughput selection platform with tunable sensitivity, a wider growth dynamic range, and the option to entirely omit exogenous NMN⁺ supplementation to select for enzymes that function with the physiologically relevant levels of NMN⁺ produced in vivo. SEQ ID NO:9 provides the polypeptide sequence of a GDH Ortho. In a further embodiment, the disclosure provides for a recombinantly engineered polypeptide that is at least 80%, 85%, 90%, 95%, 96%, 97%, 98%, or 99% identical to the sequence presented in SEQ ID NO:9. In yet a further embodiment, the recombinantly engineered polypeptide used in methods disclosed herein has the sequence presented in SEQ ID NO:9.

Polypeptides that promote intracellular synthesis of the noncanonical cofactor generally have activity to produce noncanonical cofactors from naturally occurring substrates (e.g., nicotinamide, pyridine mononucleotide, etc.). Examples of such polypeptide(s) includes those that have NAD⁺ synthase and/or nicotinate phophoribosyltransferase activities.

Genes that are utilized by the microorganism for natural glucose metabolism, include phosphoglucose isomerase pgi gene, 6P-gluconate dehydrogenase gnd gene, and glucose-6-phosphate 1-dehydrogenase zwf gene. The genes may be disrupted using stand techniques in the art, include mutagenesis and use of gene editing systems (e.g., CRISPR). In a further embodiment, additional genes may be disrupted to minimize noncanonical cofactor degradation. Examples of such genes include, but are not limited to, the pncC gene and the nadR gene.

It should be understood that the growth selection platform of the disclosure is not just limited to the unnatural cofactor exemplified in the studies presented herein (e.g., NMN⁺), and in-fact can be engineered to have greater catalytic efficiencies for additional noncanonical cofactors, such as 1-phenyl-1,4,-dihydronicotinamide (PNA⁺), 1-benzyl-1,4-dihydronicotinamide (BNA⁺), 1-(4-hydroxyphenyl) 1,4-dihydronicotinamide (HPNA⁺), 1-methyl-1,4-dihydronicotinamide (MNA⁺), nicotinamide flucytosine dinucleotide (NFCD⁺), nicotinamide mononucleoside (NR⁺), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile. For example, the studies presented herein show that some activity for BNA⁺. Regarding modifying the growth selection platform disclosed herein to utilize a nonnatural cofactor other than NMN⁺, such modifications can include changing the specificity of ortho GDH disclosed herein for another nonnatural cofactor using the directed evolution methods presented in the studies presented herein, and possibly, disrupting different genes involved with the degradation of this nonnatural cofactor.

As shown in the studies presented herein, the growth selection platform of the disclosure exhibited good NMNH-recycling activity, with tunable selection stringency by titrating NMN⁺ supplementation. Moreover, as shown in the studies presented herein, the growth selection platform of the disclosure can function as a self-sufficient platform that operates without any addition of exogenous NMN⁺, depending on how the heterologous polypeptides are expressed. Thus, the growth selection platform of the disclosure is ideally suited as a high throughput screening platform for identifying/selecting variants capable of leveraging physiologically relevant levels of NMN⁺ produced in vivo.

There are multiple advantages of compositions, methods, and kits of the disclosure. For example, the growth selection platform can be leveraged to rapidly screen for noncanonical-utilizing enzymes, which may enable more economical and scalable biotransformations. Noncanonical-utilizing enzymes identified using the growth selection platform of the disclosure are generally highly proficient catalysts in recycling noncanonical cofactors due to their superior TTN, ability to maintain turnover at low noncanonical concentrations, and capability to recycle other noncanonical cofactors.

By using the growth selection process described herein, variants of Lactobacillus pentosus NADH oxidase (Lp Nox) were generated herein. These variants, in comparison to wild type LP Nox, effectively and efficiently recycle NMNH. Water-forming NADH oxidases (Nox) are commonly utilized in cofactor regeneration processes to sustain NAD(P)⁺ concentrations and minimize feedstock costs as stoichiometric input of the oxidized cofactor is economically nonviable. The wild-type Lp Nox was demonstrated to oxidize N-methyl-1,4-dihydronicotinamide (MNAH) and N-benzyl-1,4-dihydronicotinamide (BNAH), though at low catalytic efficiencies. Protein engineering could expand the cofactor scope and scale activity to levels comparable or greater than the native cofactor NADH. Using the growth-based selection platform, iterative engineering was performed to obtain a variant LP-3-EP with ~10-fold increased catalytic activity toward NMNH. In addition, this variant also exhibited ~2-4-fold increased activity toward MNAH and BNAH.

In contrast to other mutation design strategies that focused on restructuring the binding pocket near the site of catalysis at the nicotinamide ring or saturation mutagenesis strategies, the design strategy presented herein targeted more transferrable mutations near the conserved dinucleotide binding domain of Rossman fold. Through in-depth exploration of sequence space, cooperative mutations were identified that were located near the binding interface of the nicotinamide ring, surrounding the adenosine binding cleft, and at the surface of the protein. To understand the mechanistic basis for the improved activity with NMNH, experimental validation was performed to characterize the functional contribution of each individual mutation. Finally, use of molecular modeling captured flexibility changes undetectable with available structure determination methods. This illustrated how the mutations shift the ensemble of binding conformations sampled to mirror more closely those of wild-type Lp NOX with the native cofactor NADH. The discovered variants independently converged on substitutions promoting dense packing in the region where the adenosine of the native cofactor would bind and biased conformational flexibility to favor sampling catalytically relevant states. By coupling functional data and analysis of full-atom conformational dynamics, provided emergent design principles for future engineering of polypeptides with activity for noncanonical redox cofactors.

In the studies presented herein, cofactor binding was investigated in silico at the molecular level and the results were harmonized with previous engineering efforts into accessible design guidelines. Mutations in the first shell of the cofactor binding pocket can be readily interpreted through their effects on hydrophobic packing or polar interactions with the ligand. 1158S falls in this category, and its parallel appearance in Ss GDH 1192T-V306I raises the importance of positioning the Rossman pyrophosphate binding alpha helix for coordinating cofactor binding. By using the growth selection methods disclosed herein, combinatorial mutations in the adenosine pocket were investigated. From which, it was discovered LP-3 and LP-7 variants which shared dense hydrophobic packing with D177W-V240L in the vacant cleft that the larger NADH would fill. Additionally, MD analysis used to describe conformational changes, indicated that the cooperative effects of the mutations serve to rigidify the active site by reducing local loop flexibility and support hydride transfer by maintaining contact between the cofactor and FAD. WT Lp NOX with MNAH bound was unable to sample hydride transfer competent poses due to excessive loop flexibility bumping the cofactor out of the binding site. Global analysis of backbone dynamics, through free energy decomposition, further illustrated that the mutations caused mutant Lp NOX with MNAH bound to sample similar catalytically competent states as the wild-type Lp NOX with NADH. The mutations restored the ability of mutant Lp NOX to populate configurations that the wild-type Lp NOX with MNAH cannot access. The additional hydrophobic packing cemented the binding pocket loops and allowed MNAH to bind without interference. By mimicking the effect of having the AMP bound and stabilizing the protein at locations inaccessible by the smaller biomimetic, the enzyme is better primed for catalysis.

The disclosure further provides a cell-free system or whole-cell biomanufacturing systems to facilitate the biotransformation of a substrate into a desired product, comprising a recombinantly engineered polypeptide of the disclosure that has improved catalytic efficiency towards a noncanonical cofactor, and one or more polypeptides or proteins that encode enzymes that can use the same noncanonical cofactor in a biotransformation reaction(s).

The disclosure further provides a whole cell biomanufacturing system to facilitate the biotransformation of a substrate into a desired product, comprising a recombinant microorganism of the disclosure that has been modified to express an engineered polypeptide of the disclosure, i.e., a polypeptide that has improved catalytic efficiency towards an noncanonical cofactor and optionally express one or more polypeptides or proteins that encode enzymes that can use the same noncanonical cofactor in a biotransformation reaction(s) (see e.g., Liang et al., Metabolic engineering 39, 181-191 (2017); Machado et al., Metabolic engineering 14, 504-511 (2012); and Zhang et al., ACS synthetic biology (2018).

The disclosure further provides that the compositions and methods described herein can be further defined by the following aspects (aspects 1 to 43):
1. A recombinant microorganism comprising:
   disruption of one or more genes utilized by the microorganism for glucose metabolism;
   a first heterologous polypeptide encoding an orthogonal noncanonical cofactor-dependent glucose dehydrogenase (GDH ortho) and a glucose facilitated porin; and
   a second heterologous polypeptide encoding polypeptides which promote intracellular synthesis of the noncanonical cofactor,
   wherein the noncanonical cofactor can be found in both a reduced and a non-reduced form, and
   wherein growth of the recombinant microorganism is controlled by the availability of the noncanonical cofactor.
2. The recombinant microorganism of aspect 1, wherein the microorganism is a yeast, an archaeon, or a bacterium.
3. The recombinant microorganism of aspect 2, wherein the bacterium is selected from Escherichia Coli, Bacillus subtilis, Bacillus brevis, Bacillus megaterium, Brevibacillus choshinensis, Caulobacter crescentus, and Lactococcus lactis.
4. The recombinant microorganism of aspect 3, wherein the bacterium is Escherichia Coli.
5 The recombinant microorganism of any one of the proceeding aspects, wherein the first heterologous polypeptide is expressed from a plasmid.
6. The recombinant microorganism of any one of the proceeding aspects, wherein the second heterologous polypeptide is expressed from a plasmid.
7 The recombinant microorganism of aspect 5 or aspect 6, wherein the first heterologous polypeptide and the second heterologous polypeptide are expressed from the same plasmid.
8. The recombinant microorganism of aspect 5 or aspect 6, wherein the first heterologous polypeptide and the second heterologous polypeptide are expressed from different plasmids.
9. The recombinant microorganism of aspect 7 or aspect 8, wherein the plasmids have low to medium copy numbers in the recombinant microorganism.
10. The recombinant microorganism of any one of the proceeding aspects, wherein the second heterologous polypeptide is integrated into a chromosome of the recombinant microorganism.
11. The recombinant microorganism of any one of the proceeding aspects, wherein the GDH ortho comprises a sequence that is least 95% identical to the sequence of SEQ ID NO:11.
12 The recombinant microorganism of aspect 11, wherein the GDH ortho comprises the sequence of SEQ ID NO: 11.
13. The recombinant microorganism of any one of the proceeding aspects, wherein the glucose facilitated porin comprises a sequence that is least 98% identical to the sequence of SEQ ID NO: 14.
14. The recombinant microorganism of aspect 13, wherein the glucose facilitated porin comprises the sequence of SEQ ID NO: 14.
15. The recombinant microorganism of any one of the proceeding aspects, wherein the polypeptides that promote intracellular synthesis of the noncanonical cofactor comprise polypeptide(s) for NAD⁺ synthase and/or nicotinate phophoribosyltransferase.
16. The recombinant microorganism of aspect 15, wherein the polypeptide for NAD⁺ synthase has a sequence that is at least 99% identical to the sequence of SEQ ID NO: 12.
17. The recombinant microorganism of aspect 16, wherein the polypeptide for NAD⁺ synthase comprises the sequence of SEQ ID NO: 12.
18. The recombinant microorganism of aspect 15, wherein the polypeptide for nicotinate phophoribosyltransferase has a sequence that is at least 99% identical to the sequence of SEQ ID NO:13.
19. The recombinant microorganism of aspect 18, wherein the polypeptide for nicotinate phophoribosyltransferase comprises the sequence of SEQ ID NO: 13.
20. The recombinant microorganism of any one of the proceeding aspects, wherein the one or more genes utilized by the microorganism for glucose metabolism that are disrupted comprises phosphoglucose isomerase pgi gene, 6P-gluconate dehydrogenase gnd gene, and/or glucose-6-phosphate 1-dehydrogenase zwf gene.
21. The recombinant microorganism of aspect 20, wherein the phosphoglucose isomerase pgi gene encodes a phosphoglucose isomerase having the sequence of SEQ ID NO: 15;
   wherein the 6P-gluconate dehydrogenase gnd gene encodes a 6P-gluconate dehydrogenase having the sequence of SEQ ID NO: 17; and
   wherein the glucose-6-phosphate 1-dehydrogenase zwf gene encodes a glucose-6-phosphate 1-dehydrogenase having the sequence of SEQ ID NO: 16.
22. The recombinant microorganism of any one of the proceeding aspects, wherein the microorganism further comprises disruption of one of more genes to minimize noncanonical cofactor degradation.
23. The recombinant microorganism of aspect 22, wherein the one of more genes to minimize noncanonical cofactor degradation comprises pncC gene, and/or nadR gene.
24. The recombinant microorganism of aspect 23, wherein the pmcC gene encodes a nicotinamide-nucleotide aminohydrolase having the sequence of SEQ ID NO:18; and wherein the nadR gene encodes a nicotinamide-nucleotide adenylyltransferase having the sequence of SEQ ID NO: 19.
25. The recombinant microorganism of any one of the proceeding aspects, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl) 1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.
26. The recombinant microorganism of aspect 25, wherein the noncanonical cofactor is NMN.
27. The recombinant microorganism of any one of the proceeding aspects, wherein the recombinant microorganism further comprises a third heterologous polypeptide that encodes a recombinantly engineered polypeptide that utilizes the reduced form of the noncanonical cofactor; and wherein expression of the recombinantly engineered polypeptide enhances the growth of the recombinant microorganism.
28. The recombinant microorganism of aspect 27, wherein the growth of the recombinant microorganism is influenced by the specificity of the recombinantly engineered polypeptide for the reduced form of the noncanonical cofactor, wherein the higher the specificity of the recombinantly engineered polypeptide for the reduced form of the noncanonical cofactor correlates with a faster growth rate of the recombinant microorganism.
29. The recombinant microorganism of aspect 27 or aspect 28, wherein the third heterologous polypeptide is expressed from a plasmid.
30. The recombinant microorganism of aspect 29, wherein the plasmid has high copy numbers in the recombinant microorganism.
31. The recombinant microorganism of any one of aspects 27 to 30, wherein the recombinantly engineered polypeptide has oxidoreductase activity.
32. A growth selection process to screen recombinantly engineered polypeptides that have activity for the reduced form of a noncanonical cofactor, the growth selection process comprising:
   culturing a recombinant microorganism of any one of aspects 27 to 31 using a first selection criteria to identify recombinantly engineered polypeptides that have activity for the reduced form of a noncanonical cofactor by identifying recombinant microorganisms that exhibit increased growth and/or growth rate;
   optionally, culturing the recombinant microorganism using additional selection criteria, wherein the additional selection criteria make it more, and more, challenging for the recombinant microorganisms to grow without having activity for the reduced form of a noncanonical cofactor.
33. The growth selection process of aspect 32, wherein a plurality of recombinantly engineered polypeptides is screened using the growth selection process, wherein each of the plurality recombinantly engineered polypeptides vary from each other by having a different polypeptide sequence.
34. The growth selection process of aspect 33, wherein the polypeptide sequences for each of the recombinantly engineered polypeptides vary by length and/or by composition.
35. The growth selection process of aspect 33, wherein the plurality of recombinantly engineered polypeptides are variants of a wild type polypeptide sequence and comprises 1, 2, 3, 4, 5, 6, 7, 8, 9, 10, 11, or 12 introduced amino acid substitutions in comparison to the wild type polypeptide sequence.
36. The growth selection process of any one of aspects 32 to 35, wherein the growth selection process is carried out using agar plates and identifying colonies which grow faster and/or larger than other colonies indicating that the recombinantly engineered polynucleotides have greater efficiencies for the reduced form of the noncanonical cofactor.
37. The growth selection process of any one of aspects 32 to 36, wherein the recombinantly engineered polypeptides have oxidoreductase or oxidase activity.
38. The growth selection process of aspect 37, wherein the recombinantly engineered polypeptides have sequence that is at least 98% identical to a polypeptide sequence of an oxidoreductase or an oxidase from a microorganism.
39. The growth selection process of aspect 38, wherein the oxidoreductase or the oxidase is selected from NADH Oxidase, NADPH Oxidase, and XenA.
40. A recombinantly engineered polypeptide that has a sequence that is at least 98% identical to a sequence of SEQ ID: NO 2, 3, 4, 5, 6, 7, 8, 9 or 10 and which has activity for the reduced form of a noncanonical cofactor but has limited to no activity for the natural cofactor.
41. The recombinantly engineered polypeptide of aspect 40, wherein the recombinantly engineered polypeptide comprises the sequence of SEQ ID NO:4, 5, 7 or 10.
42. An isolated polypeptide that encodes the recombinantly engineered polypeptide of aspect 40 or aspect 41.
43. A vector or plasmid which comprises the isolated polypeptide of aspect 42.

### EXAMPLES

In order that the present disclosure may be more fully understood, the following examples are set forth. It should be understood that these examples are for illustrative purposes only and are not to be construed as limiting this disclosure in any manner.

Media and Growth Conditions. Cloning was carried out with E. coli XL⁻¹ blue (see Table 1). All E. coli were cultured in 2×YT containing 16 g/L tryptone, 10 g/L yeast extract and 5 g/L NaCl unless otherwise noted. M9 Wash Buffer consisted of 1 mM MgSO₄, 0.1 mM CaCl₂), trace metal mix A5 with Co (H₃BO₃ 2860 μg/L, MnCl₂·4H₂O 1810 μg/L, ZnSO₄ 7H₂O 222 μg/L, Na₂MoO₄, 2H₂O 390 μg/L, CuSO₄, 5H₂O 79 μg/L, Co(NO₃)₂·6H₂O 49 μg/L), and BD Difco M9 salts (Na₂HPO₄6.78 g/L, KH₂PO₄ 3 g/L, NaCl 0.5 g/L, NH₄Cl 1 g/L). M9 Selection Media and Expression Media were prepared as described in Black et al. (Nature Chemical Biology 16:87-94 (2020)). M9 Selection Media shared the same composition as the M9 Wash Buffer with the inclusion of 20 g/L D-glucose, 1 mM nicotinamide, 0.1 mM IPTG, and 0.05% (w/v) arabinose, 200 mg/L ampicillin, 50 mg/L for kanamycin, and 50 mg/L for spectinomycin. For solid media M9 Selection Plates, 15 g/L agar was added in addition to M9 Selection Media composition. Expression media consisted of 2×YT media supplemented with 20 g/L D-glucose, 1 mM nicotinamide, and antibiotics as above. NMN⁺ was added to a concentration of 0, 0.5, 2, or 5 mM to M9 Selection Media as needed and specific concentrations are noted in the selection experiment methods below. All strains were cultured at 37 °C with 250 RPM. agitation and concentrations for antibiotic selection were 100 mg/L for ampicillin, 50 mg/L for kanamycin, and 50 mg/L for spectinomycin, unless otherwise noted.

TABLE 1
Strains and plasmids used in this study

| | Description |
|---|---|
| Strains | |
| XL-1 blue | Cloning strain |
| BL21 | Protein expression strain |
| (DE3) | |
| BW25113 | E. coli F-, DE(araD-araB)567, lacZ4787(del)::rrnB-3, |
| | LAM-, rph-1, DE(rhaD-rhaB)568, hsdR514 |
| DH10β | Electrotransformation strain |
| MX103 | BW25113 ΔpncC Δpgi Δzwf ΔnadR Δgnd::kan |
| MX501 | BW25113 ΔpncC Δpgi Δzwf ΔnadR Δgnd |
| MX502 | BW25113 ΔpncC Δpgi Δzwf ΔnadR Δgnd + pLM106 + |
| | pLS502 |
| MX503 | BW25113 ΔpncC Δpgi Δzwf ΔnadR Δgnd ΔpoxB:: kan- |
| | P_{LlacO1} Ft NadEV |
| Plasmids | |
| pCP20 | Temperature-inducible yeast Flp recombinase gene |
| | controlled by λcIts857 in a temperature-sensitive |
| | replicon |
| pKD46 | Ori 101 repA101ts PBAD gam-bet-exo Amp^{r} |
| pQElac | Amp^{r}; ColE1 ori; P_{LlacO1}. Expression vector |
| pZA | Kan^{r}; p15A ori; P_{LlacO1}. Expression vector |
| pRSF | Spec^{r}; RSF ori; P_{BAD}. Expression vector |
| pEK102 | pQElac 6xHis Pp XenA, Amp^{r} |
| pSM103 | pQElac, Ft NadEV - Zm glf - Re gntK, Amp^{r} |
| pSM106 | pRSF Bs GDH I195R-A93K-Y39Q-S17E, Spec^{r} |
| pLS401 | pQElac 6xHis Lb Nox, Amp^{r} |
| pLS402 | pQElac 6xHis TP Nox (Lb Nox G159A-D177A-A178R- |
| | M179S-P184R), Amp^{r} |
| pLS501 | pQElac 6xHis Pp XenA D116E, Amp^{r} |
| pLS502 | pZAlac, Ft NadEV - Zm glf, Amp^{r} |
| pLS503 | pRSF Bs GDH I195R-A93K-Y39Q-S17E - Zm glf, Spec^{r} |
| pLS504 | pQElac 6xHis Lp Nox, Amp^{r} |
| pLS505 | pQElac 6xHis Lp Nox I158S, Amp^{r} |
| pLS506 | pQElac 6xHis Lp Nox I158S-D177X-G178X-V240X, Amp^{r} |
| pLS507 | pQElac 6xHis Lp Nox I158S-D177W-V240L, Amp^{r} |
| pLS508 | pQElac 6xHis Lp Nox I158S-D177W-G178E-V240L, Amp^{r} |
| pLS509 | pQElac 6xHis Lp Nox I158S-D177W-G178E-V240L-P362H- |
| | V395L, Amp^{r} |
| pLS510 | pQElac 6xHis Lp Nox D177W-V240L, Amp^{r} |
| pLS511 | pQElac 6xHis Lp Nox D177W-G178E-V240L, Amp^{r} |
| pLS512 | pQElac 6xHis Lp Nox D177W-G178E-V240L-P362H- |
| | V395L, Amp^{r} |

Abbreviations indicate source of genes:
Bs, Bacillus subtilis;
Ft, Francisella tularensis;
Lb, Lactobacillus brevis;
Lp, Lactobacillus pentosus;
Pp, pseudomonas putida;
Re, Ralstonia eutropha H16;
Zm, Zymomonas mobilis

Strain Construction. Strain MX501 was generated by eliminating kanamycin resistance carried by E. coli strain MX103 as described in Black et al. Strain MX501 served as the template strain for MX502 and MX503. Plasmid pCP20 was used to eliminate kanamycin resistance in between integrations/knockouts performed.

Strain MX502 was constructed by co-transforming plasmids pLM106+pLS502 into strain MX501. After transformation, cells were plated and grown overnight at 37 °C. The following day, a single colony was used to inoculate a 4 mL 2× YT overnight culture. To generate dense electro-competent cells of E. coli MX502 for control experiments and for library transformations, cells were cultured in 200 mL SOB medium at 30 °C with shaking at 250 RPM until OD₆₀₀ₙₘ reached 0.4-0.6. The culture was chilled on ice for 15 minutes and the cells were pelleted at 4 °C, 4000×g. The cells were washed at 4 °C three times with 40 mL sterile 10% glycerol in water. After which, cells were resuspended with 500 μL sterile, ice-cold 10% glycerol in water and aliquoted for storage at −80 °C.

Strain MX503 was constructed by chromosomal integration of the P_{lacO1} Ft NadEV operon using the lambda red homologous recombination plasmid pKD46. The integration cassette was constructed by amplifying the operon from plasmid pLS103 from the P_{LlacO1} promoter to the stop codon of Ft NadV. SOE PCR was used to generate and amplify a fragment carrying a kanamycin cassette adjacent to the P_{lacO1} Ft NadEV cassette using KOD Xtreme Hot Start DNA Polymerase (Novagen). Homologous arms of the kanamycin-P_{LlacO1}-Ft NadEV expression cassette (1000 bp) were designed for integration within the site of non-essential gene pyruvate oxidase. Integration was confirmed by MyTaq Colony PCR (Bioline) and sequencing of region. The protocol to generate dense electro-competent cells of MX503 matched the protocol for MX502 with appropriate antibiotics.

Plasmid Construction. All PCR fragments were generated using PrimeSTAR Max DNA Polymerase (TaKaRa) unless otherwise noted. After PCR and gel extraction, all gene fragments were inserted into the pQElac vector backbone which contains a 6×His tag at the N-terminus (ColEl ori, Amp′), using Gibson isothermal DNA assembly method unless otherwise noted.

Plasmid pLS502 was constructed using plasmid pSM103 as a template. A forward primer binding at the start codon of Ft NadE paired with a reverse primer binding at the end of Zm glf was paired to amplify to amplify Ft NadEV-Zm glf insert. This fragment was inserted into the pZAlac vector backbone (p15A ori, Kan^{r}).

Plasmid pLS503 was constructed using pSM103 and pSM106 as templates. RBS-Zm glf insert was generated by pairing a forward primer binding at the RBS (ribosomal binding site) adjacent to Zm glf on pSM103 and a reverse primer binding at the end of Zm glf using pSM103 as the template. RSF-Bs GDH 1195R-A93K-Y39Q-S17E, (RSF ori, Spec′) backbone was generated by pairing a reverse primer binding at the end of Bs GDH and a forward primer binding at the terminator region following the end of Bs GDH using pSM106 as the template. These fragments were assembled to form pLS503 using Gibson Assembly.

Protein Expression and Purification. Lp Nox wild type or variants (see Table 1) are expressed with E. coli BL21 (DE3) with plasmids inoculated in 2× YT media with 200 mg L⁻¹ ampicillin. Expression was carried out by inoculation of 20 mL 2× YT media supplied with antibiotics and an overnight culture incubated at 30 °C, 250 RPM with an OD₆₀₀ₙₘ of 0.072. Cells were grown at 37 °C in 50 mL conical tubes, and the cultures were induced with 0.5 mM IPTG at an OD₆₀₀ₙₘ of 0.6-1.0. The 20 mL culture was then incubated at 27 °C and 250 RPM for 24 h. After 24 h, cells were harvested for protein extraction (centrifugation at 4 °C, 3000 RPM, 20 min). Cell pellets were resuspended in Zymo His-Binding buffer (Zymo Research Corporation) supplied with 0.15 mM FAD, 1 μL/mL DNase (Sigma Aldrich) and 1 mM InSolution AEBSF (Sigma Aldrich). The resuspended cell mixture was added to 0.5 mL of 0.1 mm glass beads (Biospec) and homogenized using a benchtop homogenizer (MP Biomedicals). Cell debris was separated from the crude extract by centrifugation at 4 °C and 15,000 RPM for 5 min. Protein purification was performed using His-Spin Protein Miniprep (Zymo Research Corporation) according to the manufacturer's instructions. The concentrations of purified protein were quantified by a Bradford assay using BSA as a standard. Purified protein was stored in aliquots with 20% (v/v) glycerol at −80 °C.

Standard Selection Protocol of Lp NOX Libraries and Control Plasmids into MX502. After dense electro-competent cells were prepared, 20 μL of library DNA was added to 200 μL competent cells. The cell-DNA mixture was added to four ice chilled 1 mm gap electroporation cuvettes. Plasmids pEK102 (XenA WT) and pLS504 (Lp Nox WT) served as positive and negative controls, respectively. For each control, 1 μL of plasmid was added per 50 uL of cells. Cells were electroporated at 2 kV, 129 Ω, 50 μF, resistance 2.5 kV. 200 μL of SOC was immediately added after shocking and the solution was transferred to a microcentrifuge tube. After another two repeats, cells were rescued at 37 °C with shaking for 1 hour.

After rescue, library cultures were transferred into 40 mL of Expression Media in a 250 mL baffled flask. Control cultures were transferred into 10 mL of expression media in loosely capped 50 mL conical tubes. Serial dilution of the library culture was plated on 2× YT agar plates with antibiotics. After incubation at 37 °C overnight, colonies formed were counted to estimate transformation efficiency. After performing serial dilutions, the remaining cells were grown at 30 °C with shaking for 3-4 h before induction with 0.5% (w/v) arabinose and 0.1 mM IPTG. After expressing for 3-4 h, the cells were collected. To prepare cells for the selection condition, 1 mL of each culture was pelleted in 2 mL microcentrifuge tubes and washed three times with 1 mL M9 Wash Buffer described above.

Selection of Lp NOX Library pLS506 and pLS509. The washed library culture was diluted to a final concentration of ~10⁵ cells/mL in M9 Wash Buffer. 100 μL of this suspension was plated on fifteen M9 Selection Plates. Plates were incubated at 30 °C and monitored periodically for 10 days. After 10 days, two colonies from each plate and colonies from the control plates were streaked onto fresh M9 Selection Plates to isolate variants and validate growth. Of these 30 colonies, only 14 grew significantly on the new plate. After 6 days, plates were evaluated for growth and 10 colonies with diverse growth phenotypes were selected for sequencing (see Table 3) and NMNH activity characterization. Colonies were cultured in 2× YT liquid media overnight and plasmids were extracted using QIAprep Spin Miniprep kit (Qiagen) to yield plasmids pLS507 and pLS508.

Error-Prone Library Construction. The Lp Nox Error-Prone PCR Library was cloned using the GeneMorph II Random Mutagenesis Kit (Agilent) to generate approximately 2 random mutations per template, variant LP-3. Using 150 ng of template DNA, the gene insert was amplified via error prone PCR according to manufacturer's instructions. A panel of starting template material (150 ng, 200 ng, 250 ng) was initially evaluated to tune range of mutations observed. To construct the Lp Nox error-prone insert library, forward and reverse primers containing BamHI-HF and Xbal digestion sites, respectively, were used in the PCR reaction. The gel purified insert fragment and plasmid pLS504 (backbone) were separately digested with BamHI-HF and Xbal (NEB). Both digestion products were again gel purified and appropriate length fragments were assembled by ligation, desalted, and transformed into the 1:1 mixture of ElectroMAX DH10B cells (Invitrogen) and 10% sterile glycerol using electroporation. Cells were rescued with SOC at 37 °C for 1 h and added to 10 mL 2× YT medium with 100 mg ml-1 ampicillin. The error prone library size was determined by serial dilution of culture on 2× YT agar plates with ampicillin at 37 °C overnight. Six single colonies were randomly picked and cultured to extract plasmids. The sequencing results confirmed the average random mutations was 2.33 per variant. The remaining liquid culture was incubated at 37 °C and 250 RPM for 10 hours, before extraction of the library DNA.

Error Prone Library Selection of Lp Nox. Following standard selection protocol of Lp Nox stated above, the washed library culture was diluted to 10⁶ cells/mL and 10⁵ cells/mL. 50 μL of each concentration of diluted cell suspension was plated on five M9 selection plates. Plates were incubated at 30 °C and monitored periodically for 11 days. Then, 59 single colonies from library plates, and colonies from the control plates were streaked onto fresh M9 selection plates to isolate variants and validate growth. Colonies picked from library plates were selected based on colony size and growth compared to template controls. After 8 days, plate growth was evaluated, showing 22 colonies with advanced growth phenotypes compared with the control template. These were selected for sequencing and NMNH activity.

XenA activity assays. XenA specific activity assays were performed in a solution containing 200 mM potassium phosphate buffer at pH 7.5, 0.2 mM reduced redox cofactor, and 5 mM ketoisophorone at 37 °C. Purified XenA was spiked into the solution to initiate the reaction. Reaction progression was monitored by overserving the change in light absorption at 340 nm, corresponding to the rate of consumption of the reduced cofactor.

NMNH generation. NMNH generation was performed in 20 mL reaction volumes in a 50 ml conical tube containing 200 mM Tris-Cl at pH 8.0, 100 mM sodium chloride, 3 mM potassium chloride, 200 mM glucose, and 20 m MNMN⁺. The reaction was initiating by spiking 500 ng of purified Bacillus subtilis glucose dehydrogenase I195R8. The reaction was incubated at 30 °C until reaction progression stopped, approximately 6 h. Reaction progression was monitored by observing the increase in light absorption at 340 nm, corresponding to the reduction of NMN⁺ to NMNH. Then, the solution was filtered through a 15 kD cutoff filter to remove the protein. To purify the NMNH, the flowthrough was mixed with HPLC-grade acetonitrile to a final concentration of 70% acetonitrile, and the solution was passed through a Phenomenex Strata NH₂ (2 g/12 mL, 55 μm, 70 Å) solid phase extraction column. The column was washed twice with 4 mL of 70:30 acetonitrile:water, then twice with 4 mL of 50:50 acetonitrile:water, twice with 4 mL of 30:70 acetonitrile:water, twice with 4 mL of nanopurified water, and finally twice with 4 mL of methanol. To elute the NMNH from the column, 2 mL of a solution containing 50:50 ammonium hydroxide:methanol was added to the column and incubated for 3 minutes before eluting. The elution step was repeated until no NMNH remained in the flow through, as monitored by light absorption at 340 nm. The elution fractions containing NMNH were pooled and aliquoted in 1 mL volumes into clean tubes. The solutions were dried over air and stored at −80 °C until use.

Lp Nox Enzymatic Assays and Kinetic study with NMNH, NAD(P) H. The Lp Nox activity (see FIG. 4A) was determined by the change in light absorption at 340 nm at 37 °C in 50 mM Tris-Cl buffer pH 7.0. The reactions were started by adding the purified protein to the prewarmed 37 °C assay mixture containing 0.3 mM of respective cofactor. The absorbance change at 340 nm for NAD(P) H and NMNH was detected using a SpectraMax M3 microplate reader. For kinetics parameter determination (see Table 2), cofactor concentrations were varied from 0 to 2 mM and the results were fitted to the standard Michaelis-Menten equation or Lineweaver-Burk equation to acquire catalytic efficiencies (k_{cat}/Kₘ) when the cofactor concentration tested could not saturate the enzyme activity or during the presence of substrate inhibition. Cofactor concentration over 2 mM could not be measured accurately on M3 spectrometer due to detector absorbance saturation at 340 nm.

TABLE 2
Apparent kinetics parameters of Lp Nox variants

| | | | | k_{cat}/Kₘ |
|---|---|---|---|---|
| Enzyme | Cofactor | Kₘ (mM) | k_{cat}(s⁻¹) | (mM⁻¹s⁻¹) |
| WT Lp Nox | NADH | 0.024 ± 0.005 | 21 ± 1 | 844 ± 4 |
| (wild type) | NADPH | 1.9 ± 0.2 | 18 ± 2 | 9.3 ± 0.2 |
| | NMNH | ND^{a} | ND^{a} | 2.3 ± 0.1 |
| LP-7 | NADH | 1.7 ± 0.2 | 47 ± 4 | 28 ± 1 |
| (I158S-D177W- | NADPH | 0.89 ± 0.04 | 27 ± 2 | 31 ± 1 |
| V240L) | NMNH | ND^{a} | ND^{a} | 18 ± 1 |
| LP-3 | NADH | 1.9 ± 0.1 | 31 ± 1 | 17 ± 1 |
| (I158S-D177W- | NADPH | 1.1 ± 0.1 | 28 ± 1 | 26 ± 1 |
| G178E-V240L) | NMNH | ND^{a} | ND^{a} | 10 ± 1 |
| LP-3-EP | NADH | ND^{b} | ND^{b} | 49 ± 2.0^{c} |
| (I158S-D177W- | NADPH | ND^{b} | ND^{b} | 51 ± 3^{c} |
| G178E-V240L- | NMNH | ND^{a} | ND^{a} | 23 ± 1 |
| P362H-V395L) | | | | |

Reactions were performed in 50 mM Tris-HCl buffer pH 7.0 at 37 °C. Details can be found in the Methods. Errors shown are the standard deviation of two or three independent measurements.
ND, not determined.
^{a}Enzyme could not be saturated with the cofactor concentrations tested.
^{b}Enzyme exhibited inhibition by the cofactor that cannot be fit with the standard substrate inhibition rate equation.
^{c}Estimated based on kinetics under lower cofactor concentrations before the inhibition was observed.

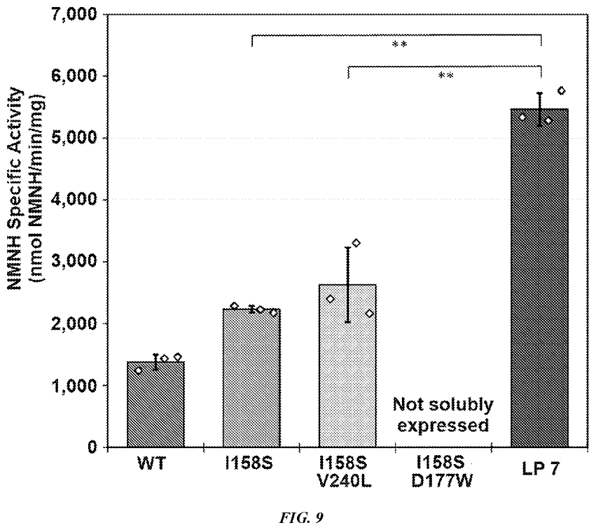

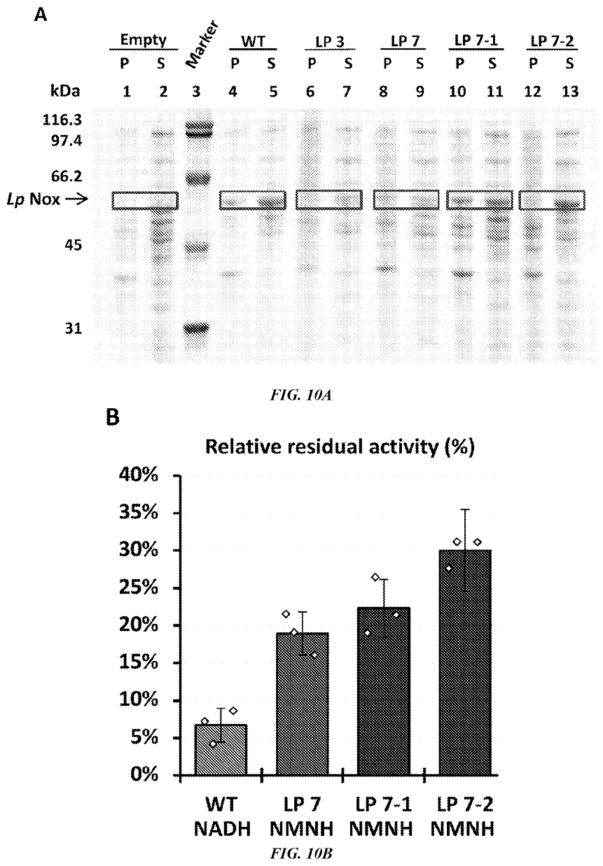

Lp Nox variants thermostability characterization. Thermostability (see FIG. 10B) was tested by incubating the enzyme at 50 °C for 23 h and measuring residual enzymatic activity relative to each sample's initial measurement following the protocol descried above. Proteins were cooled down to assay temperature, 37 °C, from 50 °C before initializing the reaction.

Protein solubility analysis. WT Lp Nox, variants, and empty plasmid (pSM105) were expressed following the Protein Expression and Purification protocol. Cell pellets were resuspended to an OD₆₀₀ nm of 4.5 with Zymo His-Binding buffer mixture and subsequently homogenized. Lysates were clarified by centrifugation. The supernatant was pipetted to a separate tube, and insoluble pellets were resuspended in the same volume of buffer as before lysis. 0.7 μL was taken from either the insoluble pellet resuspension or the supernatant to load on 12% SDS-PAGE (BIO-RAD) for the solubility test (see FIG. 10A).

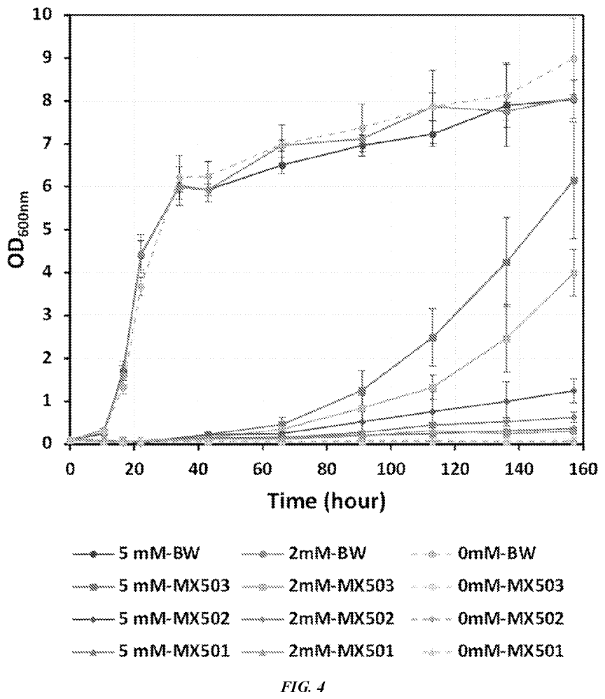

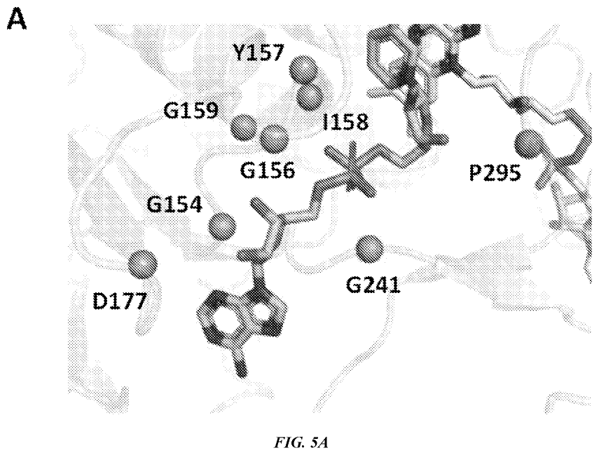

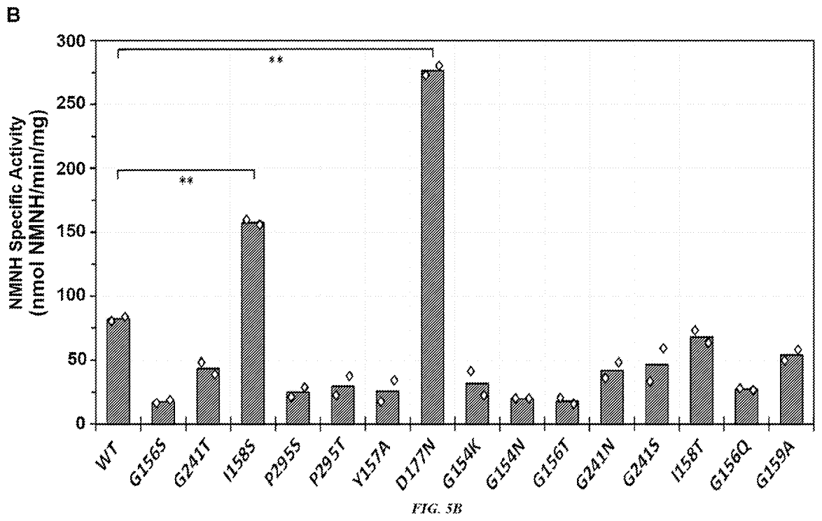

Activity Assays with other Noncanonical Cofactors. Stock solutions of MNAH (Cayman Chemical) and BNAH were dissolved to 30 mM in pure DMSO before use. The final assay mixture contained 50 mM Tris-Cl buffer pH 7.0 and 0.3 mM of cofactors to a final DMSO concentration of 1.0%. At 37 °C, the variants' activities (see FIG. 5) were measured by the change in light absorption at 360 nm. An extinction coefficient 8360 nm of 6646.43 M⁻¹ cm⁻¹ for MNAH, 8360 nm of 7254 M⁻¹ cm⁻¹ for BNAH were used to correlate rate of change in light absorption to reaction rate.

### BNAH Synthesis.

1-Benzylnicotinamide (BNA⁺) was chemically synthesized as described by Guarneri et al. (ChemCatChem 12:1368-1375 (2020)) with the following modifications: the scale was 20 mmol of nicotinamide and 20 mmol of 1-benzylbromide in 15 mL of acetonitrile, and no diethyl ether was added to assist precipitation of the product. The title compound was afforded at an 89% isolated yield (5.24 g, 24 mmol). ¹H NMR (400 MHZ, D20): δ 9.35 (s, 1H), 9.07 (ap d, J=6.2, 1H), 8.89 (ap d, J=8.2 Hz, 1H), 8.18 (ap dd, J=8.2, 6.2 Hz, 1H), 7.49 (s, 5H), 5.89 (s, 2H).

1-Benzyl-1,4-dihydronicotinamide (BNAH) was chemically synthesized as described by Guarneri et al. with the following modifications: the scale was 6 mmol of 1-benzylnicotinamide, 18 mmol of sodium dithionite, and 12 mmol of sodium carbonate; the reaction procedure took place under an argon atmosphere and the resulting product was sufficiently pure so as to not require recrystallization in a methanol-water mixture. The product was dried over calcium sulfate instead of phosphorous pentoxide. The title compound was afforded at a 60% yield (1.41 g, 11 mmol). ¹H NMR (400 MHz, DMSO-d₆): δ 7.32-7.19 (m, 5H), 7.10 (s, 1H), 5.70-5.67 (m, 3H), 4.71-4.68 (m, 1H), 4.23 (s, 2H), 3.11 (m, 2H).

Modeling Lp NOX variants. The homology model of Lp Nox was constructed with the I-TASSER webserver. The binding poses of the FAD and NADH ligands were set by aligning the homology model with the related Lb Nox crystal structure (PDB: 5VN0), which shares 65% sequence identity, and transferring the coordinates for the ligands. Final refinement for the binding modes was carried out through Rosetta relaxation using the FastRelax protocol to produce an energy minimized model with optimized protein-ligand interactions. The model of the MNAH ligand was downloaded from the PubChem database, and docking was performed by superposition of the nicotinamide ring and carboxamide group with the corresponding atoms in NADH to match the crystal binding pose. A similar binding mode is expected to be necessary for catalysis since MNAH is a truncated version of the native cofactor. Modeling preparations and docking of NMNH were done using Rosetta. Models for the variant Lp Nox were produced with Rosetta. Each amino acid substitution was made individually with the MutateResidue mover, then Monte Carlo sampling steps were performed where all residues or ligands within 6 Å of any mutation were allowed to sample alternative rotamer states and backbone dihedrals or torsions to optimize intermolecular interactions. Backbone lever-arm effects were avoided by only making changes in isolated, contiguous residue segments at each move. Local regions were minimized with the EnzRepackMinimize mover or altered by pivoting a segment using the Backrub mover. 1,000 decoys for each mutant were generated, and the sample with the most favorable total Rosetta score (an aggregate of hydrophobic, electrostatic, and geometric terms) was selected as the representative model. Example source code are available at GitHub.

TABLE 5
Accession codes of crystal structures and genes used in this study.

| Item | Full name | Accession code |
|---|---|---|
| Ec Pgi | Glucose-6-phosphate isomerase | Uniprot ID: P0A6T1 |
| Ec Zwf | NADP(+)-dependent glucose-6-phosphate | Uniprot ID: P0AC53 |
| | dehydrogenase | |
| Ec Gnd | 6-phosphogluconate dehydrogenase | Uniprot ID: P00350 |
| Zm Glf | Glucose facilitated diffusion porin | Uniprot ID: P21906 |
| Ec PncC | NMN aminohydrolase | Uniprot ID: P0A6G3 |
| Ec NadR | NMN adenylyltransferase | Uniprot ID: P27278 |
| Ft NadE | NAD(+) synthase | Uniprot ID: Q5NFH5 |
| Ft NadV | Nicotinate phosphoribosyltransferase | Uniprot ID: A0A0E3A662 |
| Bs Gdh | Glucose 1-dehydrogenase | Uniprot ID: P12310 |
| Pp XenA | NADH: flavin oxidoreductase/NADH oxidase | Uniprot ID: Q9R9V9 |
| Lb Nox | FAD-dependent oxidoreductase [Levilactobacillus | PDB: 5VN0 |
| | brevis] | |
| TP Nox | triphosphopyridine nucleotide oxidase | doi: 10.1038/nchembio.2454 |
| Lp Nox | FAD-dependent oxidoreductase | Uniprot ID: F6IXY6 |

Molecular dynamics simulations. Molecular dynamics simulations (MD) simulations of the Lp Nox variants were completed with PMEMD from the AMBER20 package utilizing the ff14sb force field and 8 Å Particle Mesh Ewald real space cutoff. Ligand partial charges were determined with the Restrained Electrostatic Potential (RESP) method at the HF/6-31 G* level using Gaussian09, and other parameters were taken from the General Amber Force Field (GAFF). Protonation states of titratable residues were determined with the H++webserver. The TLEAP program was utilized to solvate the complexes with TIP3P water molecules in a truncated octahedron with 10 Å buffer and 150 mM salt concentration with Na⁺/Cl⁻ counterions. The Lp Nox systems were minimized in two stages, first with 2,500 steps of steepest descent and 2,500 steps of conjugate gradient where all non-hydrogen solute atoms were restrained with a 20 kcal mol⁻¹ Å⁻² force to relieve solvent clash. The second stage minimization to remove solute steric clashes was run with the same cycle settings and restraints removed. Heating from 0 K to 300 K was performed over 0.5 ns with 10 kcal mol⁻¹ Å⁻² restraints on all non-hydrogen solute atoms under NPT conditions at 1 atm pressure with Langevin thermostat and 1 fs timestep. Solvent density equilibration was run over 5 ns with 5 kcal mol⁻¹ Å⁻² restraints on all solute atoms and an unrestrained 10 ns equilibration using 2 fs timestep to clear remaining structural artifacts followed the heating stage. Production MD trajectories were each carried out for 400 ns with 2 fs timestep, SHAKE restraints on hydrogens, NVT ensemble, Langevin thermostat with collision frequency 1.0 ps⁻¹, and periodic boundary conditions. Two independent replicates were run for the three samples: WT Lp Nox with NADH, WT Lp Nox with MNAH, and LP 3-EP 1158 with MNAH.

Conformational flexibility and free energy structural analysis. Three metrics were utilized to compare the effects of the mutations on Lp Nox conformational dynamics: binding pocket loop flexibility as measured by Root Mean Square Deviation (RMSD) of alpha carbon residues located on loops regions, hydride transfer potential through evaluation of the distance between the nicotinamide ring C4 and FAD N5, and free energy landscape decomposition based on dimensionality reduction with Principal Component Analysis (PCA) of the protein alpha carbon dihedrals. The replicate trajectories were aligned to the reference Rosetta models using alpha carbons for all analysis. Binding pocket loops were defined as residues lining the Rossman fold where the native cofactor NADH is bound. The three regions selected include the positions: 153-157, 177-188, and 238-242. Hydride transfer was categorized into active and inactive binding poses with a 4.5 Å distance cutoff. This was selected as during the simulation the cofactors had the ability to fully dissociate from the protein and sampling large distances would disproportionately influence aggregate values. A cofactor distance of 5 Å would have no material difference in biological effect from 10 Å as both would indicate that the cofactor is too far to participate in catalysis. Phi and psi angles for all alpha carbons were parsed to characterize global conformational dynamics. The data was preprocessed through standard scaling to center the means at zero and variances at one, then transformed through PCA. The two-dimensional free energy landscape was plotted based on the first two principal components, and free energy values were calculated from the density of normalized histogram counts with energy minimum shifted to zero. Structural analysis was completed with the scikit-learn, PyEMMA, pytraj, and PyMOL software packages.

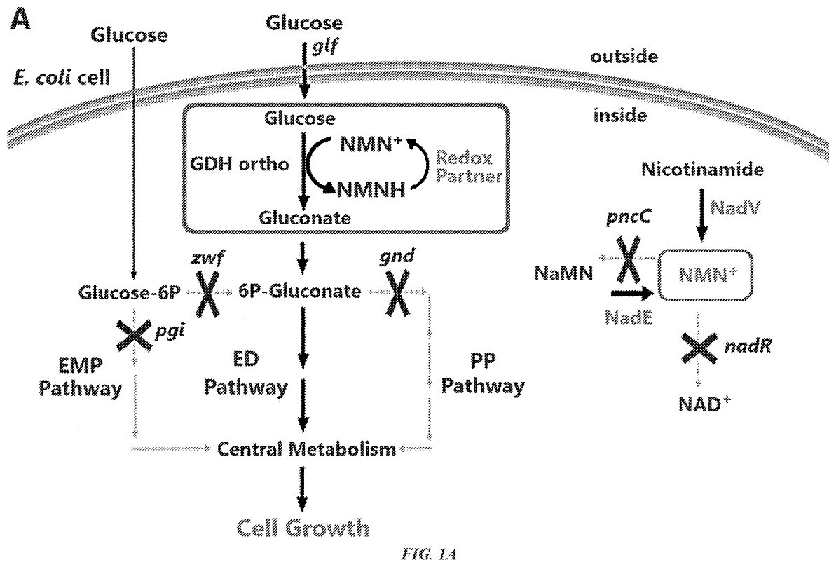

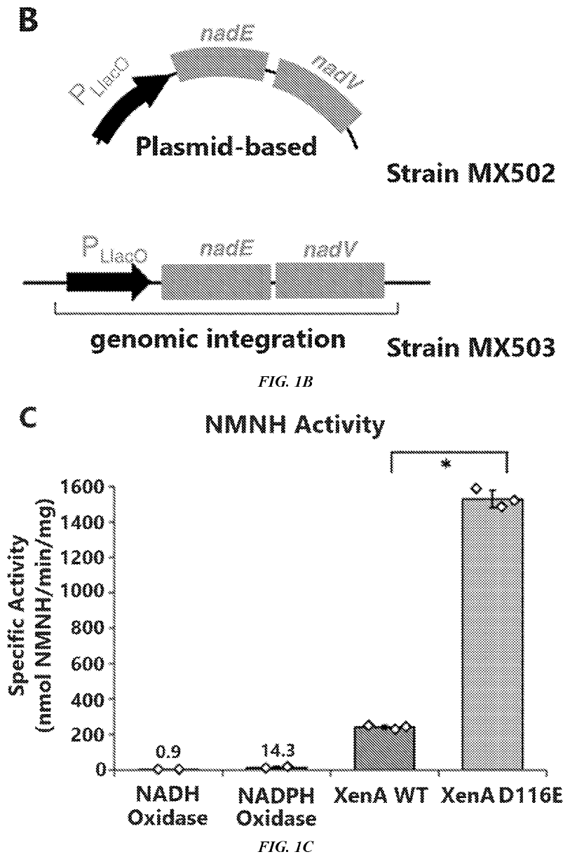

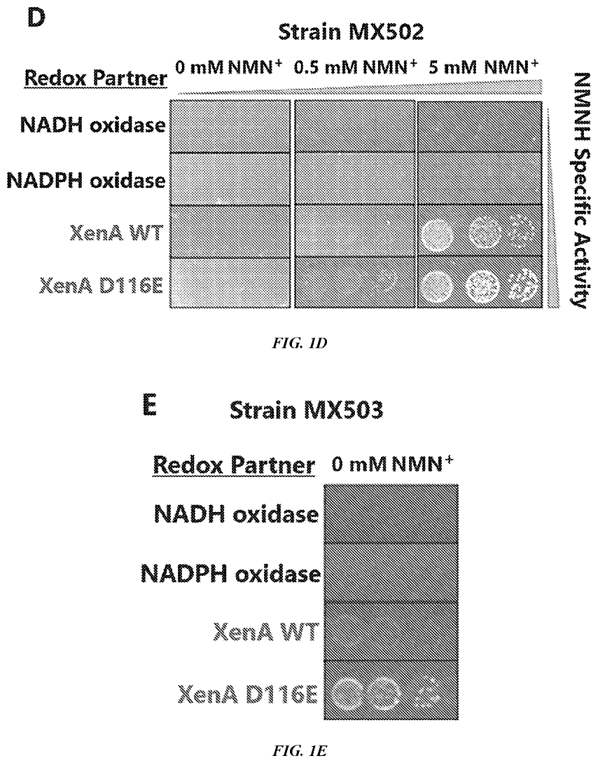

Development and validation of NMNH-dependent growth selection. The selection platform is based on an E. coli strain that was engineered to disrupt natural glucose metabolism. The deletion of phosphoglucose isomerase pgi and glucose-6-phosphate 1-dehydrogenase zwf gene eliminated conventional glycolytic routes in the Embden-Meyerhof-Parnas (EMP) and pentose phosphate (PP) pathways, respectively. The resulting strain is unable to grow in minimal glucose media without the installation of an alternate pathway for life-essential central metabolism (see FIG. 1A). Previously, it was demonstrated that glucose catabolismin this strain can be facilitated by the Entner Doudoroff (ED) pathway through the expression of an orthogonal NMN⁺-dependent glucose dehydrogenase (GDH ortho) and the cycling of the corresponding oxidized cofactor NMNH by an active redox partner.

In this system, conversion of glucose to gluconate is controlled by the availability of NMN⁺. As NMN⁺ is reduced to NMNH, its accessibility becomes limited and requires a redox cycling partner to oxidize NMNH and regenerate the supply, enabling restored carbon flux through the ED pathway. While the initial iteration of this strain demonstrated a clear dependence of growth on the presence of NMN⁺, GDH ortho, and an NMNH-dependent cycling partner, its practicality as a selection platform was limited. To improve utility, the strain was reconfigured to (1) expand the dynamic range of growth, insuring high and low activity redox partners are distinguishable, and (2) express the redox partner gene from a standalone plasmid, to allow interchangeability of different engineering targets. The modified selection strain, MX502 (see Table 1), carries two plasmids (pLM106+pLS502) which possess the genes for initial glucose entry (GDH ortho and a glucose facilitated diffusion porin from Z. mobilis, Zm Glf), and NMN⁺ intracellular synthesis (NAD⁺ synthase, Ft NadE, and Nicotinate phophoribosyltransferase, Ft NadV) (see FIG. 1A-B). These two supporting plasmids have low to medium copy numbers to minimize growth burden. A third, high-copy plasmid is used to introduce various redox partners.

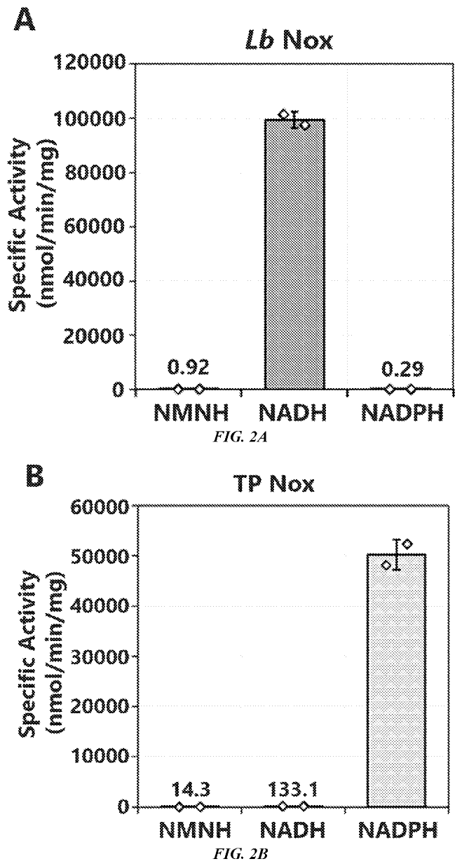

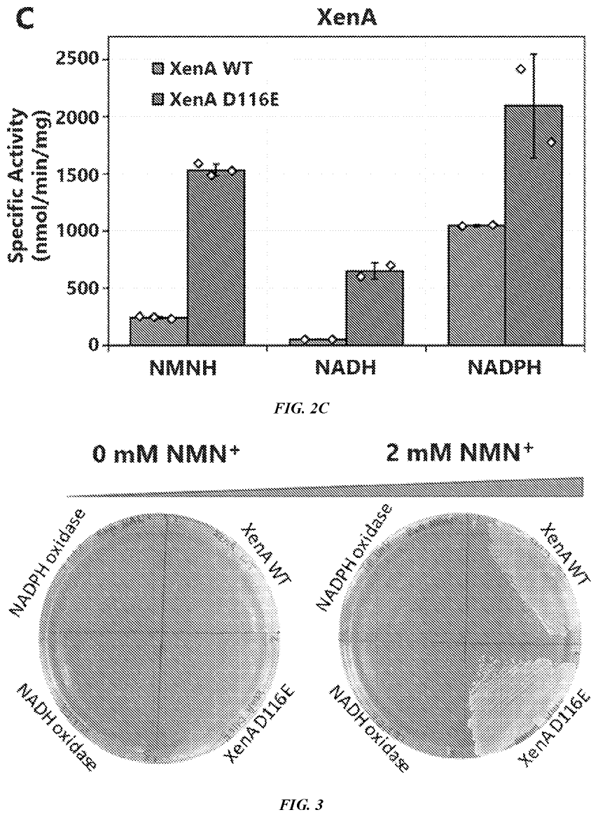

To benchmark the optimized strain's sensitivity to NMNH activity, four individual redox partners were tested: an NADH-specific oxidase from L. brevis (Lb Nox) (see FIG. 2A), an NADPH-specific oxidase (triphosphopyridine nucleotide oxidase, TP Nox) (see FIG. 2B) an enoate reductase (XenA) from P. putida with broad cofactor specificity including NMNH (see FIG. 2B), and a mutant XenA D116E that demonstrates enhanced activity towards all cofactors compared to the wild-type XenA (see FIG. 1C and FIG. 2C). After transformation into MX502, cell growth was characterized on solid selection media with varying NMN⁺ supplementation (see FIG. 1D). Growth of cells expressing the NADH, and NADPH-dependent oxidase was negligible, which highlighted the selection's high specificity in presenting only the NMNH-utilizing redox partner, but not those cycling partners specific to the native redox cofactors in the cells. Additionally, expression of NMNH active XenA facilitated growth in a dose-dependent manner with NMN⁺ supplementation, and XenA variant D116E sustained stronger growth (see FIG. 1D and FIG. 2). This growth trend aligned well with the redox partners NMNH-oxidizing activity and validated that the readout from growth can distinguish increased NMNH activity.

Development of a self-sufficient NMNH-dependent growth selection. NMN⁺ was demonstrated as an efficient and orthogonal reducing equivalent in E. coli whole cells. NMN⁺ can be synthesized intracellularly from inexpensive feedstocks using engineered metabolic pathways which eliminates the cost of cofactor supplementation and makes the whole-cell biotransformation more scalable. The challenge is to rapidly obtain NMN(H)-dependent enzymes with suitable Kₘ values to directly function in the self-sufficient, NMN⁺-synthesizing strains. Therefore, to tackle this challenge, modifications of the growth selection developed above was sought.

It was previously demonstrated that internally biosynthesized NMN⁺ can support GDH Ortho function and sustain cell growth, albeit to a much lower extent compared to exogenous NMN¹ supplementation. To promote healthier cell growth, the strain MX503 was generated that featured two primary modifications: (1) the chromosomal integration of Ft NadEV (see FIG. 1B), and (2) a reduction in the total number of plasmids required to enable growth (see Table 1). Co-transformation of the supportive plasmid pLS50X carrying both GDH ortho and Zm glf along with the redox partner panel yielded a growth profile that mirrored the trend observed in MX502, but without supplementation of NMN⁺ in the selection media (see FIG. 1E). As before, increasing NMNH specific activity from WT XenA to XenA D116E correlated with an improved capacity for growth while the expression of the NADH and NADPH oxidase did not facilitate growth. Increased size difference between colonies carrying XenA WT and D116E suggests lower NMN⁺ availability heighted the sensitivity of the selection (see FIG. 3). Notably, both selection strains MX502 and MX503 carrying XenA D116E amplified within a similar time frame with or without NMN⁺ supplementation, respectively. Though strain MX503 is not used for Lp Nox screening, its self-sufficiency can be applied to select mutants capable of serving as active redox partners to efficiently sustain NMN⁺ cycling without any supplement. The approach limits false-positive mutants that can cycle using the supplied NMN⁺ (see FIG. 3).

Development of a self-sufficient NMNH-dependent growth selection. NMN⁺ can be synthesized intracellularly from inexpensive feedstocks using engineered metabolic pathways (see Table 3), which eliminates the cost of cofactor supplementation and makes the whole-cell biotransformation more scalable. The challenge is to rapidly obtain NMN(H)-dependent enzymes with suitable Kₘ values that directly function in the self-sufficient, NMN⁺-synthesizing strains. It has been demonstrated that endogenously biosynthesized NMN⁺ can support GDH Ortho function and sustain cell growth, albeit to a much lower extent compared to exogenous NMN⁺ supplementation. To promote healthier cell growth, strain MX503 was generated which features two primary modifications: (1) the chromosomal integration of Ft NadEV (see FIG. 1B) and (2) a reduction in the total number of plasmids required to enable growth (see Table 1). Co-transformation of the supportive plasmid pLS503, carrying the genes encoding GDH Ortho and Zm Glf, and the redox partner panel plasmid, yielded a growth profile that mirrored the trend observed in MX502 without the need for supplementation of NMN⁺ in the selection media (see FIG. 1E). As before, increasing NMNH specific activity from WT XenA to XenA D116E correlated with an improved capacity for growth, while the expression of the NADH and NADPH oxidase did not facilitate growth. Increased size difference between colonies carrying XenA WT and D116E suggests lower NMN⁺ availability increased the sensitivity of the selection (see FIG. 1E, and FIG. 4).

TABLE 3
Summary on native and noncanonical cofactors advantages.

| | Cofactor | | | |
|---|---|---|---|---|
| Cofactor | class | Advantages | Disadvantages | Cost |
| NAD(P)⁺ | Native | Native biological redox | Increased cost of | ~$1500-$4500/kg |
| | | cofactor. Extensive | biological sourcing | |
| | | knowledge in engineering | | |
| | | for improved activity or | | |
| | | redox cofactor specificity | | |
| | | switches | | |
| NMN⁺ | Biologically | Truncation of NAD+ offers | Increased cost of | ~$250-$100/kg |
| | sourced | more structural similarity | biological sourcing | |
| | noncanonicals | compared to other | | |
| | | noncanonical redox | | |
| | | cofactors. | | |
| | | This similarity may improve | | |
| | | ability to engineer proteins | | |
| | | to utilize this cofactor | | |
| | | effectively relative to fully | | |
| | | synthetic cofactors. | | |
| | | Established biological | | |
| | | synthesis routes enable | | |
| | | implementation in vivo, | | |
| BNA⁺ | Synthetic | Facile chemical synthesis | High structural | ~$50-$20/kg |
| | noncanonicals | methods available leading to | deviation requires | |
| | | reduced input costs | more extensive | |
| | | | engineering | |
| | | | relative to NMN+ | |
| | | | 8. Decreased | |
| | | | stability relative to | |
| | | | NAD(H) | |
| MNA⁺ | Synthetic | Facile chemical synthesis | High structural | ~$50-$20/kg |
| | noncanonicals | methods available leading to | deviation requires | |
| | | reduced input costs | more extensive | |
| | | | engineering | |
| | | | relative to NMN⁺. | |
| | | | Decreased stability | |
| | | | relative to NAD(H) | |

Notably, even when the growth was considered rescued by the presence of XenA and XenA D116E, the selection strains still grow at a much lower rate than wild-type E. coli (see FIG. 4). This may originate from the disruption of the heavily utilized EMP glycolysis, which cannot be fully replaced by the ED pathway that naturally sustains a much lower carbon flux due to E. coli's regulatory control. Therefore, if the glucose uptake capacity through ED pathway can be enhanced through metabolic engineering or adaptive laboratory evolution, superior NMNH-oxidizing enzymes may support even faster growth allowing them to be more readily and reliably selected.

Engineering Lp Nox to utilize NMNH by rational design. With the selection systems in hand, it was next sought to develop Lp Nox as an efficient NMNH-recycling catalyst. Wild-type Lp Nox was previously shown to have a basal level of promiscuous activity toward noncanonical cofactors, which may indicate the propensity for this enzyme to receive a variety of NCBs, once engineered. The first round of rational design was based on the structural analysis of the cofactor binding pose. A total of 15 single mutations at 8 sites with the potential to form novel polar interactions, particularly with the phosphate on the NMNH tail, or reduce NAD(P) H activity by reorganizing the cofactor binding pocket, were tested (see FIG. 5).

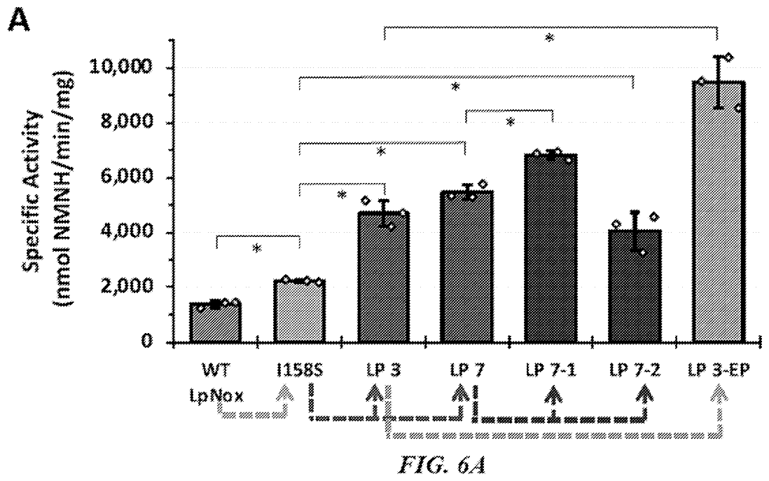

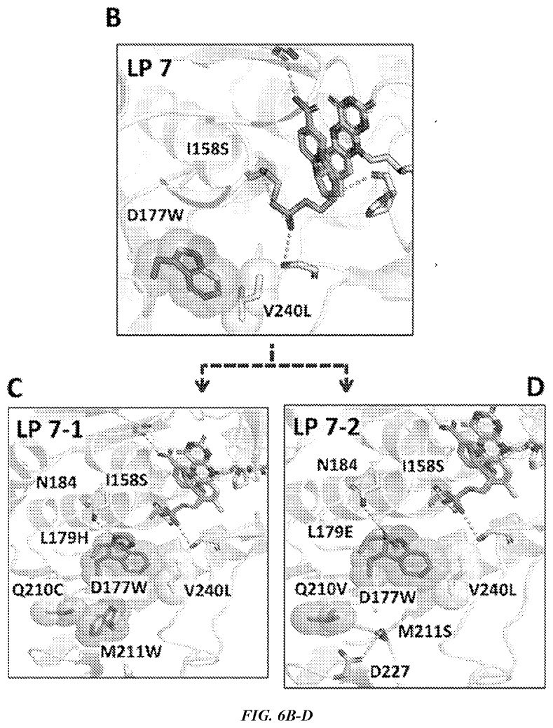

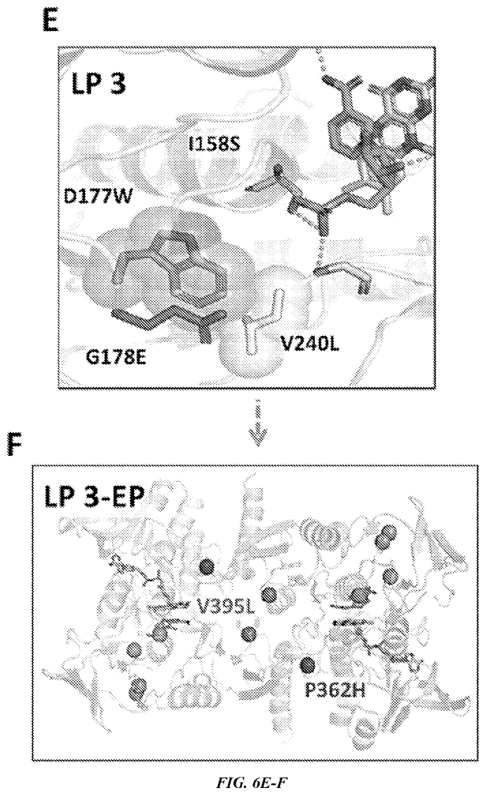

A single mutant I158S with improved specific activity for NMNH by ~2-fold compared to wild type (see FIG. 5 and FIG. 6A) was obtained, which served as the starting point for subsequent engineering efforts. It was postulated that the 1158S side chain forms a hydrogen bond with the NMNH phosphate, which is necessary to anchor NMNH and opens opportunities for further design. This design strategy has been demonstrated in our previous work. This test also shed light on the choice of sites that will be subjected to subsequent semi-rational engineering (see FIG. 5B).

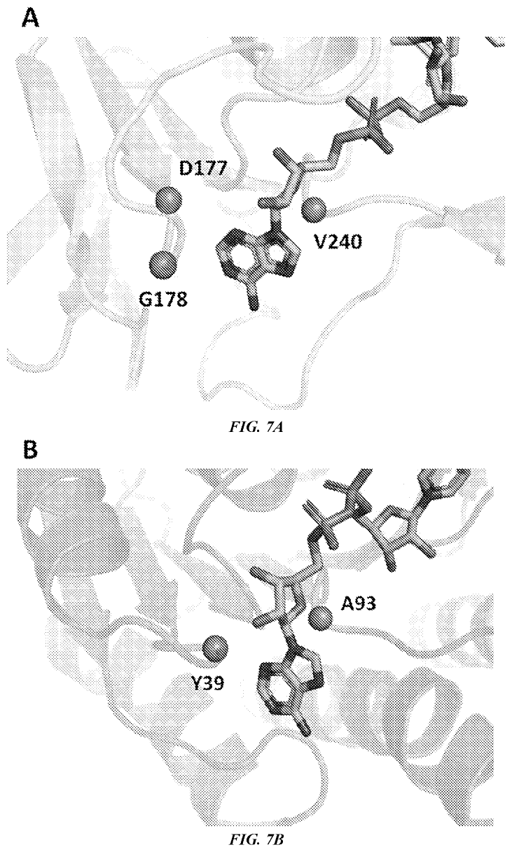

Improving Lp Nox by directed evolution through high-throughput growth selection. Based on this initial variant 1158S, the high throughput growth selection platform was leveraged to further improve NMNH activity by site-saturation mutagenesis. In this round, we targeted the first-sphere positions. These residues line the cofactor binding pocket and are analogous to the key residues that enabled our previously engineered GDH to utilize NMN⁺ (see FIG. 7). D177, G178, and V240 with NNK codons were sampled with fixed I158S.

The resulting NNK library (pLS506) was transformed into the selection strain MX502, yielding ~2.4×10⁶ independent transformants, which was sufficient to cover >10-fold of the theoretical library size of 203=8000. Selection was performed on agar plates with 20 g/L D-glucose in M9 minimal medium supplemented with 5 mM NMN⁺, at 30 °C. After incubation for 10 days, many colonies had formed on the selection plates. After subsequent colony isolation, ten were chosen for further analysis based on their fast growth, relative to other candidates.

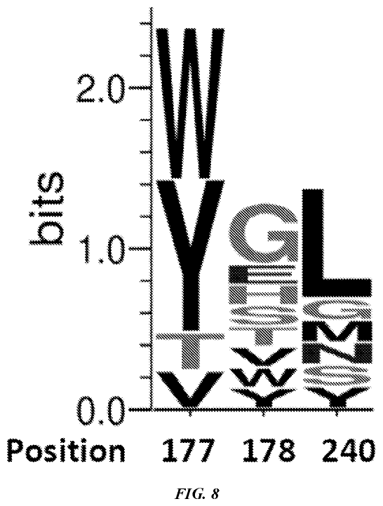

Interestingly, sequencing of the ten candidates revealed a strong consensus for bulky or hydrophobic residues at both sites 177 and 240 (see Table 4; and FIG. 8). Variant LP-7 (I158S-D177W-V240L (FIG. 6B)) was chosen to investigate further, because its mutation pattern is shared by three independently selected variants with different codons (see Table 4) suggesting strong selective pressure for this variant. LP-3 (1158S-D177W-G178E-V240L) was also chosen because it is the closest to LP-7, with just one additional mutation (see FIG. 6C). Variant LP-3 and LP-7 exhibited ~4 and ~5-fold increase of specific activity toward NMNH, respectively, compared to WT Lp Nox (see FIG. 6A).

TABLE 4
Sequences of Lp NOX variants obtained from the first selection

| | Asp 177 | | Gly 178 | | Val 240 | |
|---|---|---|---|---|---|---|
| Variant | Codon | Residue | Codon | Residue | Codon | Residue |
| WT | GAC | D | GGT | G | GTC | V |
| LP-1 | TAT | Y | CAT | H | AAT | N |
| LP-2 | TGG | Y | TAT | Y | CTT | L |
| LP-3 | TAT | W | GAG | E | CTG | L |
| LP-4 | TAT | Y | TGG | W | GGG | G |
| LP-5 | TAT | Y | TCG | S | TCT | S |
| LP-6 | GTT | V | ACG | T | ATG | M |
| LP-7 | TGG | W | GGG | G | TTG | L |
| LP-8 | TGG | W | GGT | G | CTG | L |
| LP-9 | TGG | W | GGG | G | CTG | L |
| LP-10 | ACG | T | GTT | V | TAT | Y |

The kinetic parameters of Lp Nox variants (see Table 2) were characterized to investigate the structural basis for their improved activity. Wild-type Lp Nox displayed a small amount of initial specific activity for NMNH, with a catalytic efficiency of 2.3±0.1 mM⁻¹ s⁻¹ with the linearized LP 7 contains D177W-V240L which are modeled to densely pack against each other and fill the void typically occupied by the AMP moiety in NADH (see FIG. 6B). Characterization of variants missing either one of the D177W-V240L duet supports their predicted synergy (see FIG. 9). This model suggests that the larger NADH is excluded from establishing its native binding pose through steric clash, while the smaller NMNH is minimally affected by the additional bulk. The mutation D177W also results in the loss of key polar contacts to the NADH, causing the Kₘ value for NADH to increase from 0.024±0.005 mM in the wild type to 1.7±0.2 mM with LP 7. Furthermore, the added steric hindrance may restrict the flexibility of nearby loops and result in greater pre-organization of the active site. The catalytic efficiency of LP 7 for NMNH increased ~8-fold from the wild type to 18±1 mM⁻¹s⁻¹ (see Table 2). LP 3 shares the core I158S-D177W-V240Lmutations as LP 7, and LP 3 additionally carries G178E. The increase in side chain size at the 178 position likely causes diminished loop flexibility and the larger glutamate slides underneath D177W to seal the tryptophan mutation in the adenosine cleft (see FIG. 6C). As a result, LP 3 features further diminished binding affinity for both NADH and NADPH with Kₘ values of 1.9±0.04 mM and 1.1±0.1 mM, respectively (see Table 2).

Restoring protein stability by targeting second-sphere residues. It was sought to further improve LP 7 by targeting residues L179, Q210, and M211 through another round of site-saturated mutagenesis and growth selection. These sites were suggested by Cofactor Specificity Reversal-Structural Analysis and Library Design (CSR-SALAD), a bioinformatics tool that facilitates the automated design of cofactor specificity switch between NAD(H) and NADP(H). It was postulated that the predicted, activity-boosting effects of these three sites would be translatable to engineering NMNH activity, since they are second sphere residues that do not directly contact the cofactor but may modulate the activity allosterically.

A site-saturated mutagenesis library (pLS509) with the NNK codon using LP 7 as the template was constructed and introduced in strain MX503. Growth-based selection was conducted similarly to the last round described above, with the modification that no exogenously supplied NMN⁺ was added to the selection plates when we switched to the self-sufficient selection strain. After colony isolation, we characterized the two fastest-growing variants, LP 7-1 and LP 7-2 (see FIG. 6A, D, E; and FIG. 10).

LP 7-1 represents a further improvement to LP 7 for specific activity, from ~5500 to ~6600 nmol NMNH min 1 mg-1 (see FIG. 6A). Importantly, both LP 7-1 and LP 7-2 showed markedly improved stability compared to their precursor LP 7, as evidenced by their substantially higher expression levels as soluble proteins at a comparable level to WT Lp Nox (see FIG. 10A). Additionally, LP 7-2 exhibited robust thermal stability with ~30% residual activity remaining after 23 h of heat treatment at 50 °C, which is significantly higher than its parent variant LP 7 (~19% residual activity) (see FIG. 10B). LP 7-1 (L179H-Q210C-M211W) and LP 7-2 (L179E-Q210V-M211S) share the same mutations as LP 7 with additional mutations to positions 179, 210 and 211. Rosetta modeling suggests that the additional mutations improve protein folding fitness by rigidifying flexible regions on the protein surface. The mutation L179H in LP 7-1 is predicted to form a hydrogen bond with N184, which fastens the loop that L179H resides (see FIG. 6D). L179E in LP 7-2 is predicted to have the same mode of action (see FIG. 6E). In LP 7-1, M211W and Q210C are suggested to decrease this region's backbone flexibility through the added bulk and hydrophobic interaction (see FIG. 6D). In LP 7-2, a similar effect may be mediated by the new hydrogen bond between M211S and the nearby D227 (see FIG. 6E).

The new variants' ease of soluble expression and the enhanced thermal stability will translate to lower cost in large-scale industrial applications. Furthermore, by restoring the engineered enzyme's stability, these new mutations may unlock evolutionary trajectories that involve more fitness-taxing mutations in future engineering

Random mutagenesis identified beneficial mutations more distal from active sites. In parallel to targeting the second sphere, error-prone PCR was used to search a broader protein sequence space, aimed at improving LP 3. A mutation frequency of two amino acid changes per construction was targeted. The library was introduced into selection strain MX502, yielding ~2.14×10⁶ independent transformants. Matching the growth-based selection stated previously, the error-prone PCR library was subjected to selection on media containing 0.5mMNMN+. After 11 days of monitoring, colonies growing faster than the template, indicating improved activity of cofactor cycling, were isolated. One variant LP 3-EP (1158S-D177W-G178E-V240L-P362H-V395L) was identified to exhibit an ~8-fold increase in NMNH-dependent specific activity compared to the wild-type (see FIG. 6A).

LP 3-EP accumulates two more mutations compared to LP 3, namely P362H and V395L (see FIG. 6F). The mutations P362H and V395L appear distal to the active site, ~30 Å and ~20 Å away, respectively; there is no clear rationale for their beneficial effects from the static homology models as they hang from surface regions and do not dramatically alter hydrophobic or electrostatic interactions. Overlay of the model on the biological assembly of Lb Nox (PDB: 5VN0) indicates that P362H and V395L are both positioned near the subunit interface, and they may affect cross-monomer interactions (FIG. 6F). Notably, the activity of LP 3-EP is strongly inhibited above 1 mM NADH or NADPH. However, this kinetic behavior fits poorly with the well-established substrate inhibition rate equation, suggesting that NAD(P) H may exert complex effects on this enzyme, such as excessive uncoupling which generates reactive oxygen species and damages the enzyme. Interestingly, this inhibition effect was not seen when NMNH was utilized, which may indicate better coordinated catalysis for this cofactor. LP 3-EP's catalytic efficiency for NMNH was 23±1 mM⁻¹ s⁻¹, a ~10-fold increase from the wild type and ~2.5-fold gain from LP 3 (see Table 2).

Engineered Lp Nox showed increased activity with other noncanonical cofactors. After demonstrating Lp Nox variants have increased activity towards the targeted noncanonical cofactor, NMNH, they were then tested with additional NCBs, MNAH and BNAH. These simpler noncanonical redox partners are considered more applicable in large scale thanks to their lower synthesis costs (see Table 3). In addition, by analyzing activity patterns toward structurally distinct cofactor analogs, the mechanism of cofactor-protein interaction in engineered Lp Nox can be further pinpointed.

The most active Lp Nox variant, LP 3-EP, was tested for activity using MNAH and BNAH at 0.3 mM. LP 3-EP showed ~2.5 and ~4-fold higher specific activity toward MNAH and BNAH, respectively, compared to WT Lp Nox (see FIG. 11A, B). Since the 1158S mutation in LP 3-EP was originally designed to form a hydrogen bond with the NMNH phosphate, it was postulated that this mutation might not be required for interacting with MNAH and BNAH, which have non-polar moieties attached to the nicotinamide. When the 158 position is reverted to isoleucine (pLS513), the resulting variant LP 3-EP 1158 still retains higher specific activities compared to the wild-type enzyme, especially for MNAH (see FIG. 11A). This is particularly interesting because none of the remaining mutations in LP 3-EP 1158 are predicted to directly contact MNAH given the cofactor's small size. Therefore, it was postulated that these mutations may affect the ensemble of conformations sampled by the enzyme and molecular dynamic simulations were performed to understand this effect.

The effects of mutations on Lp Nox structural flexibility. Based on the location and crowding of the D177W-G178E-V240L mutations, it was hypothesized that this group of residues, beyond simply reshaping the binding interface, would have an additional effect of increasing the rigidity of the active site. A stiff active site has been associated with increased catalytic turnover based on the principle that pre-organized residues can immediately latch onto the ligand and initiate catalysis. Active sites with a high degree of flexibility may result in frustrated binding events where the ligand encounters the protein, but the protein cannot achieve the necessary intermediate state for the reaction as the catalytic residues excessively sample irrelevant poses.

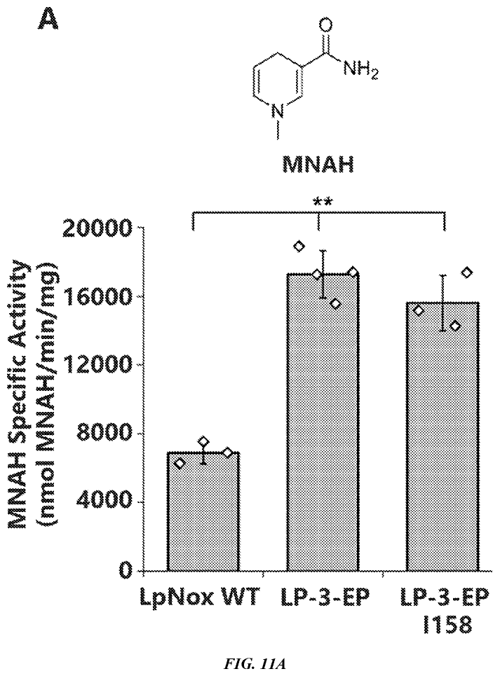

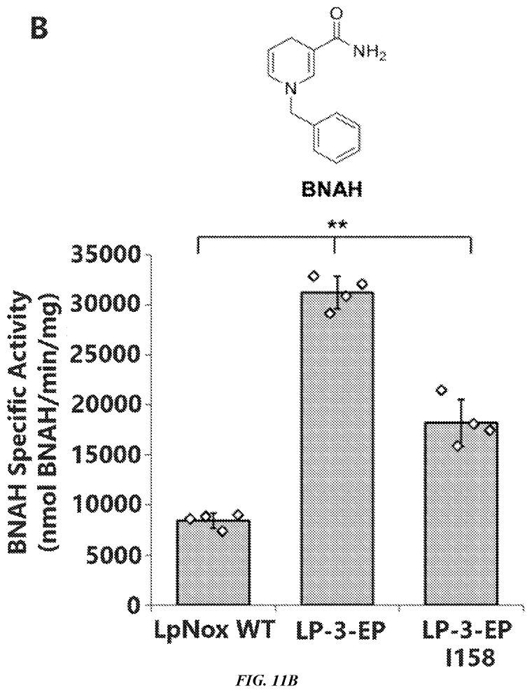

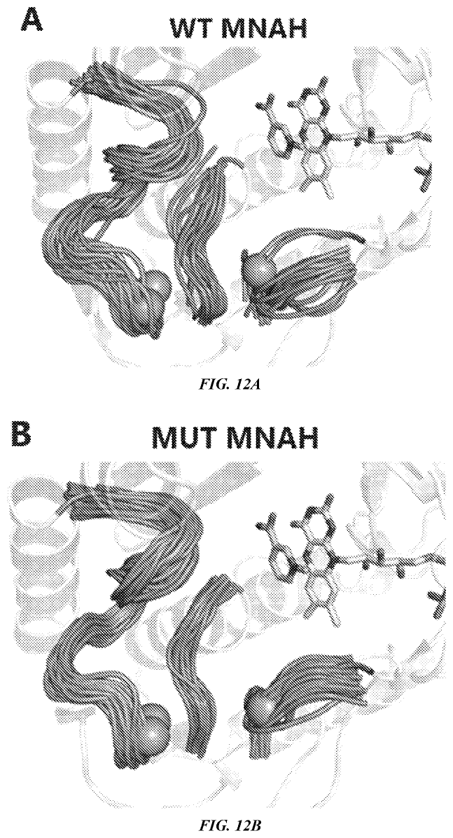

400 ns molecular dynamic (MD) simulations were performed with 2 replicates for the 3 conditions: Lp Nox wild type (WT) with NADH, WT with MNAH, and LP 3-EP 1158 (Mut) with MNAH. The rigidity of the active site loops surrounding the AMP pocket was compared through RMSD alignment to the starting homology model (see FIG. 12). Overlay of 40 randomly sampled frames from the WT with MNAH and Mut with MNAH condition illustrate how the WT protein experiences much greater plasticity when bound to this smaller cofactor with the loops having wider spread (see FIG. 12A). On the other hand, the overlay of loops in the Mut model bundle more tightly, indicating greater stability (see FIG. 12B). The differences in flexibility are quantified through comparison of the RMSD distributions for the highlighted loop residues after alignment to the starting pose (see FIG. 12C). Mut with MNAH bound had loops RMSD of 0.75±0.08 Å, nearly identical to the value found for the WT with native cofactor NADH bound of 0.74=0.10 Å. The WT with MNAH bound showed greater active site loop flexibility with 0.94±0.13 Å RMSD and prominent rightward shift in the histogram of RMSD values. The presence of the bulky residues introduced by the mutations possibly mimic the effect of having the larger NADH bound, reducing the conformational entropy of the loops surrounding the AMP pocket.

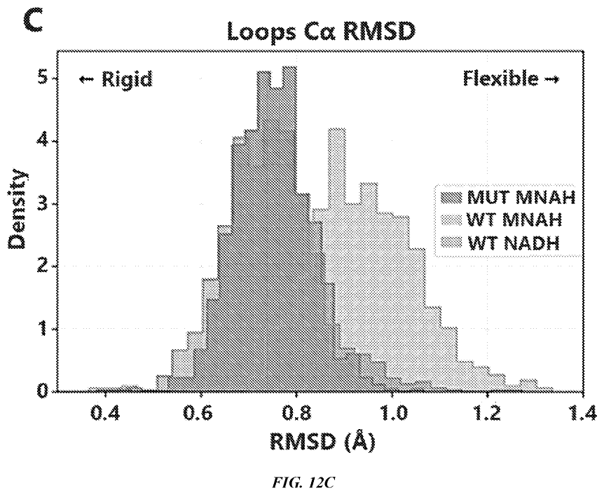

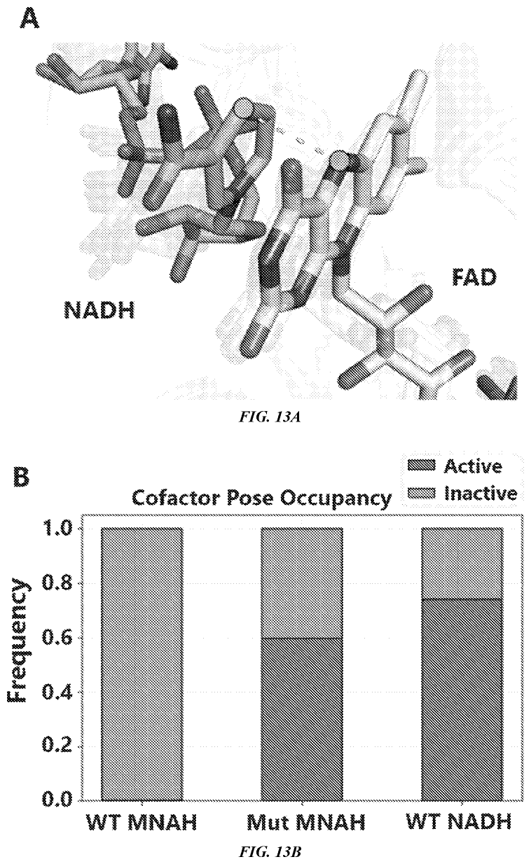

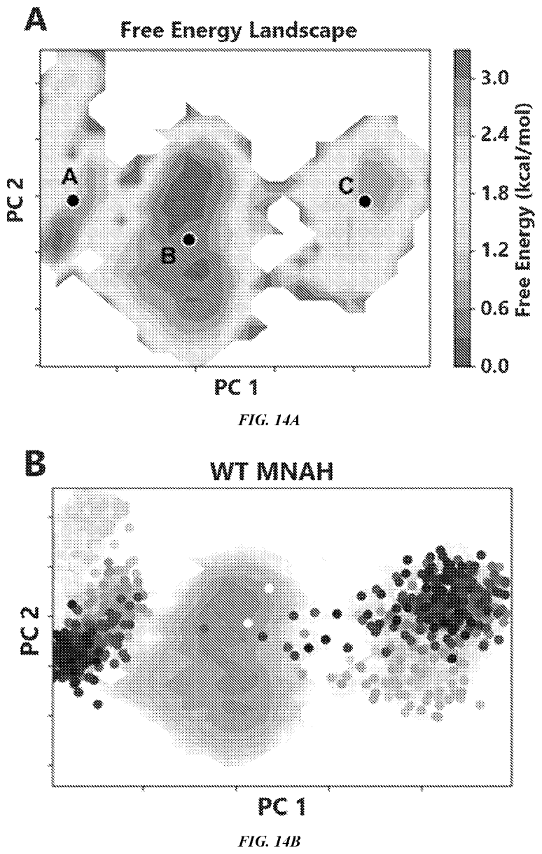

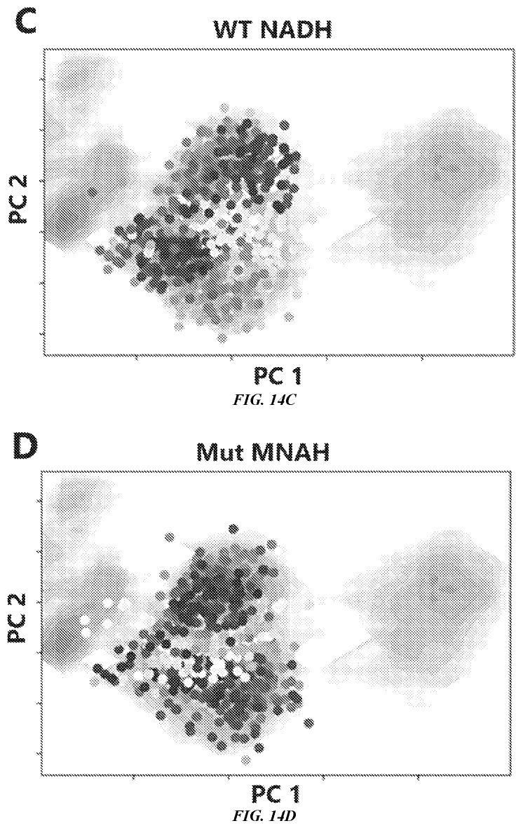

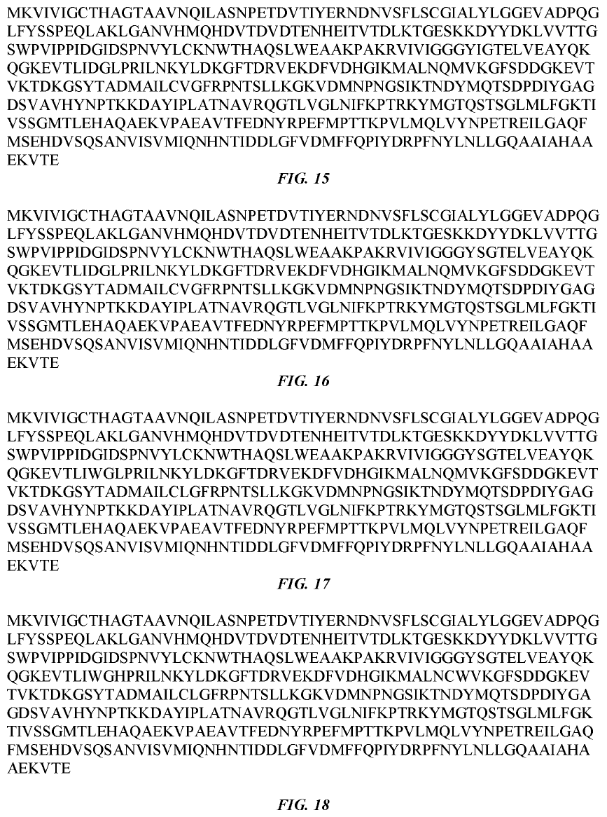

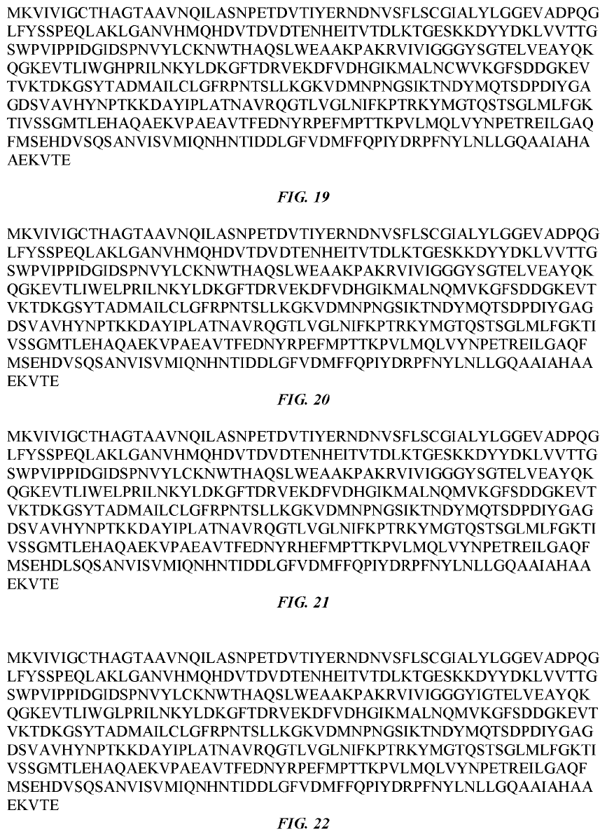

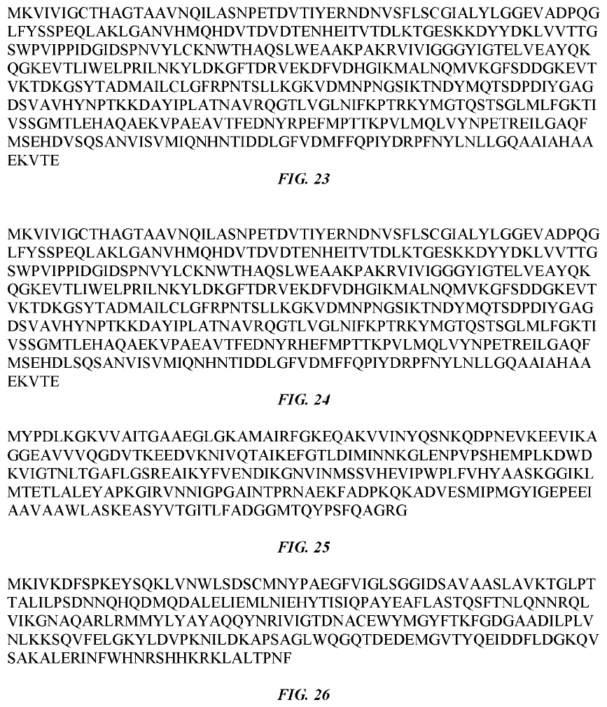

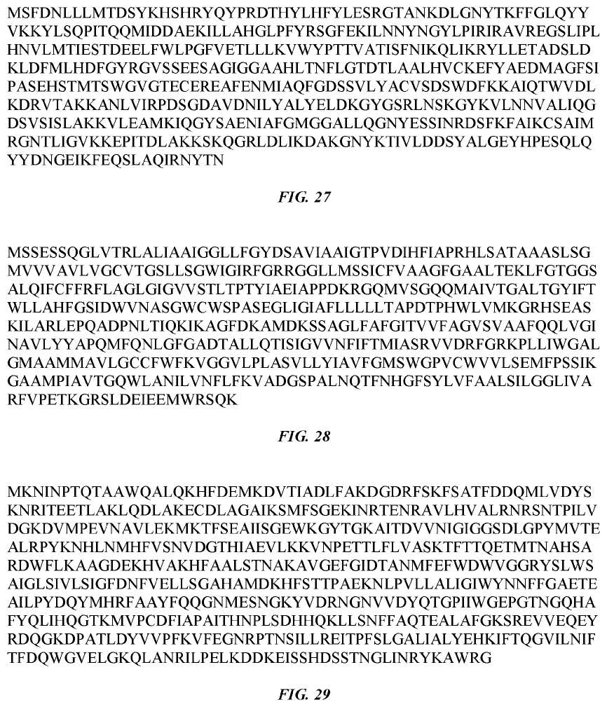

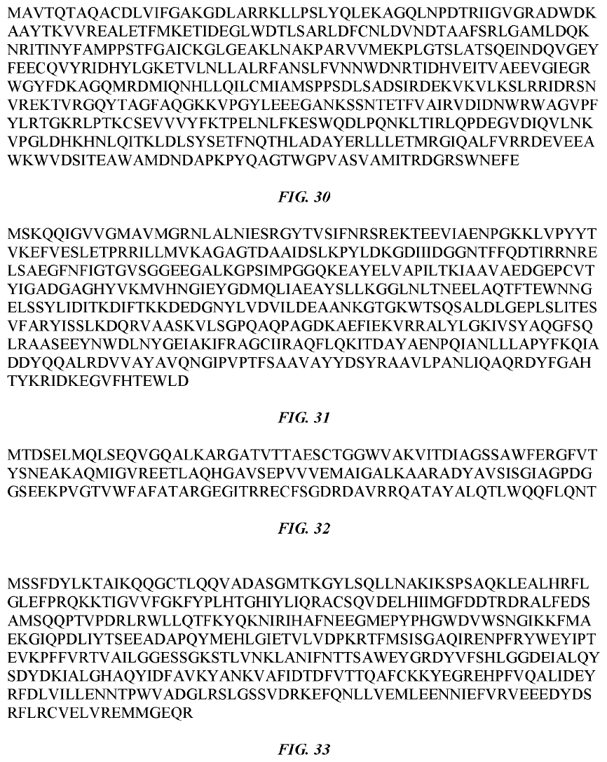

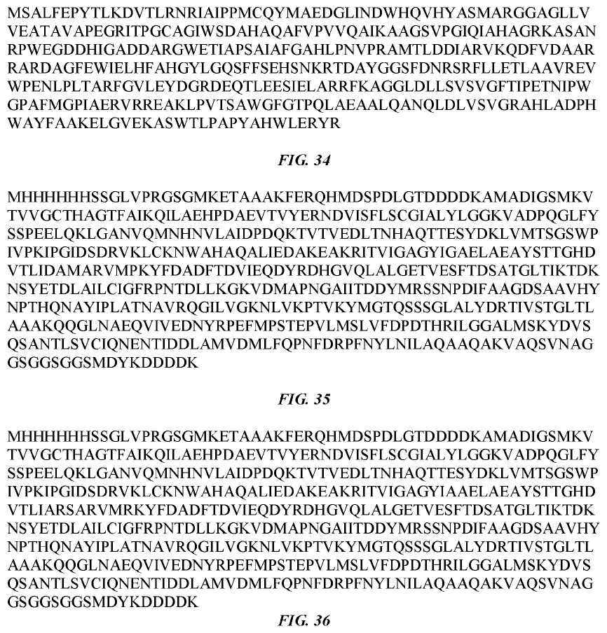

Correlation between conformational dynamics and catalytic activity. To capture the effects of altered structural flexibility in the engineered mutant on catalysis, localized effects were compared by measuring hydride transfer propensity between the nicotinamide and FAD (see FIG. 13), and global conformational dynamics (see FIG. 14). Nox catalyzed reactions are mediated by a FAD cofactor that is proposed to accept hydride transfer from NADH to become FADH2, which then passes its electrons to oxygen. Hydride transfer occurs between the nicotinamide C4 and FAD N5 (see FIG. 13A), this distance is observed to be ~3.1 Å in the crystal structure of the related Lb Nox with NADH bound (PDB: 5VN0). Inactive enzymes will rarely sample the necessary catalytic geometry, resulting in no reaction; conversely, highly active enzymes must support close contact between the nicotinamide and FAD. We defined a threshold of 4.5 Å was defined as the maximum distance between the nicotinamide C4 and FAD N5 for active catalysis since separation greater than 4.5 Å typically meant that the nicotinamide cofactor had exited the binding cavity during the MD simulation and binned the hydride transfer distances into active and inactive categories.

The distribution of cofactor poses correlates with the different levels of activity observed. WT with NADH bound maintained the active geometry for hydride transfer in 74% of the frames, and only 1% of the frames with MNAH bound (see FIG. 13B). Visual inspection of the WT trajectories suggests that the measured high loop flexibility when binding to MNAH (see FIG. 12) resulted in rattling and bumping of the cofactor out of the binding pocket, and furthermore, impeded the cofactor from returning. Mut (LP 3-EP 1158) with MNAH displayed 60% occupancy for the active pose supporting hydride transfer (see FIG. 13B). These observations suggest that dense hydrophobic packing from the D177W-G178E-V240L mutations served to lock the mobility of the binding pocket loops and allowed the MNAH to bind with reduced interference.

Enzymes sample an ensemble of backbone configurations with varying functional capabilities. Enzyme variants with enhanced activity have been discovered to sample pre-existing catalytically active states with greater frequency. Based on this observation, it was hypothesized that the discovered mutations may function by shifting the population of conformation states toward those more relevant to catalysis.

The free energy landscapes of the enzyme systems are visualized through PCA dimensionality reduction to resolve coordinates of maximal variance in the alpha-carbon dihedrals over the course of the simulations (see FIG. 14). Combined data from all MD trajectories is projected onto shared latent space defined by the first two principal components and cluster into three distinct groups. These groups each capture different local minima and are labeled by K-Means clustering as Groups A, B, and C (see FIG. 14A). Each data point represents a state, and each point is shaded by temporality with lighter colors indicating those at the start of the simulation and darker points representing data near the end. WT with MNAH occupies Groups A and C, while hardly crossing into Group B (see FIG. 14B). Most notably there is little overlap with Group B where the bulk of catalytically active states are found as indicated by the simulation of WT with NADH (see FIG. 14C). Mut (LP 3-EP 1158) with MNAH shows similar population distribution to WT with NADH by clustering in Group B (see FIG. 14D), indicating the mutations reshape the conformational landscape of Lp Nox with MNAH bound to resemble that of the WT when using its native cofactor NADH.

Certain embodiments of the invention have been described. It will be understood that various modifications may be made without departing from the spirit and scope of the invention. Other embodiments are within the scope of the following claims.

1. A recombinant microorganism comprising:
disruption of one or more genes utilized by the microorganism for glucose metabolism;
a first heterologous polypeptide encoding an orthogonal noncanonical cofactor-dependent glucose dehydrogenase (GDH ortho) and a glucose facilitated porin; and
a second heterologous polypeptide encoding polypeptides which promote intracellular synthesis of the noncanonical cofactor,
wherein the noncanonical cofactor can be found in both a reduced and a non-reduced form, and
wherein growth of the recombinant microorganism is controlled by the availability of the noncanonical cofactor.

2. (canceled)

3. The recombinant microorganism of claim 1, wherein the microorganism is selected from Escherichia Coli, Bacillus subtilis, Bacillus brevis, Bacillus megaterium, Brevibacillus choshinensis, Caulobacter crescentus, and Lactococcus lactis.

4. (canceled)

5. The recombinant microorganism of claim 1, wherein the first heterologous polypeptide is expressed from a plasmid and/or wherein the second heterologous polypeptide is expressed from a plasmid, and wherein the plasmid(s) have low to medium copy numbers in the recombinant microorganism.

6-10. (canceled)

11. The recombinant microorganism of claim 1, wherein the GDH ortho comprises a sequence that is least 95% identical to the sequence of SEQ ID NO: 11.

12. (canceled)

13. The recombinant microorganism of claim 1, wherein the glucose facilitated porin comprises a sequence that is least 98% identical to the sequence of SEQ ID NO:14.

14. (canceled)

15. The recombinant microorganism of claim 1, wherein the polypeptides that promote intracellular synthesis of the noncanonical cofactor comprise polypeptide(s) for NAD⁺ synthase and/or nicotinate phophoribosyltransferase.

16. The recombinant microorganism of claim 15, wherein the polypeptide for NAD⁺ synthase has a sequence that is at least 95% identical to the sequence of SEQ ID NO: 12.

17. (canceled)

18. The recombinant microorganism of claim 15, wherein the polypeptide for nicotinate phophoribosyltransferase has a sequence that is at least 95% identical to the sequence of SEQ ID NO: 13.

19. (canceled)

20. The recombinant microorganism of claim 1, wherein the one or more genes utilized by the microorganism for glucose metabolism that are disrupted comprises phosphoglucose isomerase pgi gene, 6P-gluconate dehydrogenase gnd gene, and/or glucose-6-phosphate 1-dehydrogenase zwf gene.

21. The recombinant microorganism of claim 20, wherein the phosphoglucose isomerase pgi gene encodes a phosphoglucose isomerase having the sequence of SEQ ID NO: 15;
wherein the 6P-gluconate dehydrogenase gnd gene encodes a 6P-gluconate dehydrogenase having the sequence of SEQ ID NO: 17; and
wherein the glucose-6-phosphate 1-dehydrogenase zwf gene encodes a glucose-6-phosphate 1-dehydrogenase having the sequence of SEQ ID NO: 16.

22. The recombinant microorganism of claim 1, wherein the microorganism further comprises disruption of the pncC gene, and/or the nadR gene to minimize noncanonical cofactor degradation.

23. (canceled)

24. The recombinant microorganism of claim 22, wherein the pmcC gene encodes a nicotinamide-nucleotide aminohydrolase having the sequence of SEQ ID NO: 18; and wherein the nadR gene encodes a nicotinamide-nucleotide adenylyltransferase having the sequence of SEQ ID NO: 19.

25. The recombinant microorganism of claim 1, wherein the noncanonical cofactor is selected from the group consisting of nicotinamide mononucleotide (NMN), 1-phenyl-1,4,-dihydronicotinamide (PNA), 1-benzyl-1,4-dihydronicotinamide (BNA), 1-(4-hydroxyphenyl) 1,4-dihydronicotinamide (HPNA), 1-methyl-1,4-dihydronicotinamide (MNA), nicotinamide flucytosine dinucleotide (NFCD), nicotinamide mononucleoside (NR), 1-butyl-1,4,5,6-tetrahydropyridine-3-carboxamide, 1-(1-benzyl-1,4,5,6-tetrahydropyridin-3-yl) ethenone, 1-benzyl-1,4-dihydropyridine-3-carboxylic acid, and 1-benzyl-1,4,5,6-tetrahydropyridine-3-carbonitrile.

26. (canceled)

27. The recombinant microorganism of claim 1, wherein the recombinant microorganism further comprises a third heterologous polypeptide that encodes a recombinantly engineered polypeptide that utilizes the reduced form of the noncanonical cofactor; and wherein expression of the recombinantly engineered polypeptide enhances the growth of the recombinant microorganism.

28. (canceled)

29. The recombinant microorganism of claim 27, wherein the third heterologous polypeptide is expressed from a plasmid that has high copy numbers in the recombinant microorganism.

30-31. (canceled)

32. A growth selection process to screen recombinantly engineered polypeptides that have activity for the reduced form of a noncanonical cofactor, the growth selection process comprising:
culturing a recombinant microorganism of claim 27 using a first selection criteria to identify recombinantly engineered polypeptides that have activity for the reduced form of a noncanonical cofactor by identifying recombinant microorganisms that exhibit increased growth and/or growth rate;
optionally, culturing the recombinant microorganism using additional selection criteria, wherein the additional selection criteria make it more, and more, challenging for the recombinant microorganisms to grow without having activity for the reduced form of a noncanonical cofactor.

33. The growth selection process of claim 32, wherein a plurality of recombinantly engineered polypeptides is screened using the growth selection process, wherein each of the plurality recombinantly engineered polypeptides vary from each other by having a different polypeptide sequence, and wherein the plurality of recombinantly engineered polypeptides are variants of a wild type polypeptide sequence and comprises 1, 2, 3, 4, 5, 6, 7, 8, 9, 10, 11, or 12 introduced amino acid substitutions in comparison to the wild type polypeptide sequence.

34-35. (canceled)

36. The growth selection process of claim 32, wherein the growth selection process is carried out using agar plates and identifying colonies which grow faster and/or larger than other colonies indicating that the recombinantly engineered polynucleotides encode polypeptide sequences which have greater efficiencies for the reduced form of the noncanonical cofactor.

37-39. (canceled)

40. A recombinantly engineered polypeptide that has a sequence that is at least 98% identical to a sequence of SEQ ID: NO 2, 3, 4, 5, 6, 7, 8, 9 or 10 and which has activity for the reduced form of a noncanonical cofactor but has limited to no activity for the natural cofactor.

41. The recombinantly engineered polypeptide of claim 40, wherein the recombinantly engineered polypeptide comprises the sequence of SEQ ID NO:4, 5, 7 or 10.

42-43. (canceled)
